SEQUENCE LISTING (26 sequences)

SEQ ID NO: 1 - RNA, 19 nt, Artificial
  siRNA for human paxillin
guguggagccuucuuuggu

SEQ ID NO: 2 - RNA, 19 nt, Artificial
  siRNA for human paxillin
accaaagaaggcuccacac

SEQ ID NO: 3 - RNA, 19 nt, Artificial
  siRNA for human paxillin
ccacacauaccaggagauu

SEQ ID NO: 4 - RNA, 19 nt, Artificial
  siRNA for human paxillin
aaucuccugguaugugugg

SEQ ID NO: 5 - RNA, 19 nt, Artificial
  siRNA for human paxillin
gaacgacaagccuuacugu

SEQ ID NO: 6 - RNA, 19 nt, Artificial
  siRNA for human paxillin
acaguaaggcuugucguuc

SEQ ID NO: 7 - RNA, 21 nt, Artificial
  siRNA for human paxillin
caacuggaaaccacacauauu

SEQ ID NO: 8 - RNA, 21 nt, Artificial
  siRNA for human paxillin
uaugugugguuuccaguuguu

SEQ ID NO: 9 - RNA, 21 nt, Artificial
  siRNA for human paxillin
gucucuuggaugaacuggauu

SEQ ID NO: 10 - RNA, 21 nt, Artificial
  siRNA for human paxillin
uccaguucauccaagagacuu

SEQ ID NO: 11 - RNA, 23 nt, Artificial
  siRNA for human paxillin
cccugacgaaagagaagccuauu

SEQ ID NO: 12 - RNA, 23 nt, Artificial
  siRNA for human paxillin
uaggcuucucuuucgucaggguu

SEQ ID NO: 13 - RNA, 25 nt, Artificial
  siRNA for human paxillin
cgggccauccuggagaacuauaucu

SEQ ID NO: 14 - RNA, 25 nt, Artificial
  siRNA for human paxillin
agauauaguucuccaggauggcccg

SEQ ID NO: 15 - RNA, 71 nt, Artificial
  Aptamer A10
gggaggacgaugcggaucagccauguuuacgucacuccuugucaauccucaucggcagacgacucgcccga

SEQ ID NO: 16 - RNA, 56 nt, Artificial
  Aptamer A10-3
gggaggacgaugcggaucagccauguuuacgucacuccuugucaauccucaucggc

SEQ ID NO: 17 - RNA, 70 nt, Artificial
  Aptamer A9
gggaggacgaugcggaccgaaaaagaccugacuucuauacuaagucuacguucccagacgacucgcccga

SEQ ID NO: 18 - RNA, 100 nt, Artificial
  Aptamer A30
uaauacgacucacuauagggaauuccgcgugugccagcgaaaguugcguaugggucacaucgcaggcacaugucaucugggcgguccguucgggauccuc

SEQ ID NO: 19 - RNA, 98 nt, Artificial
  Aptamer S6
gggagauaccagcuuauucaauuuggauggggagauccguugaguaagcgggcgugucucucugccgccuugcuauggggagauaguaagugcaaucu

SEQ ID NO: 20 - RNA, 78 nt, Artificial
  Aptamer aptTOV1
uccagagugacgcagcagaucuguguaggaucgcaguguaguggacauuugauacgacuggcucgacacgguggcuua

SEQ ID NO: 21 - DNA, 1776 bp, homo sapiens
atggacgacctcgacgccctgctggcggacttggagtctaccacctcccacatctccaaacggcctgtgttcttgtcggaggagaccccctactcatacccaactggaaaccacacataccaggagattgccgtgccaccccccgtccccccacccccgtccagcgaggccctcaatggcacaatccttgaccccttagaccagtggcagcccagcagctcccgattcatccaccagcagcctcagtcctcatcacctgtgtacggctccagtgccaaaacttccagtgtctccaaccctcaggacagtgttggctctccgtgctcccgagtgggtgaggaggagcacgtctacagcttccccaacaagcagaaatcagctgagccttcacccaccgtaatgagcacgtccctgggcagcaacctttctgaactcgaccgcctgctgctggaactgaacgctgtacagcataacccgccaggcttccctgcagatgaggccaactcaagccccccgcttcctggggccctgagccccctctatggtgtcccagagactaacagccccttgggaggcaaagctgggcccctgacgaaagagaagcctaagcggaatgggggccggggcctggaggacgtgcggcccagtgtggagagtctcttggatgaactggagagctccgtgcccagccccgtccctgccatcactgtgaaccagggcgagatgagcagcccgcagcgcgtcacctccacccaacagcagacacgcatctcggcctcctctgccaccagggagctggacgagctgatggcttcgctgtcggatttcaagatccagggcctggagcaaagagcggatggggagcggtgctgggcggccggctggcctcgggacggcgggcggagcagccccggagggcaggacgagggagggttcatggcccaggggaagacagggagcagctcaccccctggggggcccccgaagcccgggagccagctggacagcatgctggggagcctgcagtctgacctgaacaagctgggggtcgccacagtcgccaaaggagtctgcggggcctgcaagaagcccatcgccgggcaggttgtgaccgccatggggaagacgtggcaccccgagcacttcgtctgcacccactgccaggaggagatcggatcccggaacttcttcgagcgggatggacagccctactgtgaaaaggactaccacaacctcttctccccgcgctgctactactgcaacggccccatcctggataaagtggtgacagcccttgaccggacgtggcaccctgaacacttcttctgtgcacagtgtggagccttctttggtcccgaagggttccacgagaaggacggcaaggcctactgtcgcaaggactacttcgacatgttcgcacccaagtgtggcggctgcgcccgggccatcctggagaactatatctcagccctcaacacgctgtggcatcctgagtgctttgtgtgccgggaatgcttcacgccattcgtgaacggcagcttcttcgagcacgacgggcagccctactgtgaggtgcactaccacgagcggcgcggctcgctgtgttctggctgccagaagcccatcaccggccgctgcatcaccgccatggccaagaagttccaccccgagcacttcgtctgtgccttctgcctcaagcagctcaacaagggcaccttcaaggagcagaacgacaagccttactgtcagaactgcttcctcaagctcttctgctag

SEQ ID NO: 22 - protein, 591 aa, homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 23 - protein, 557 aa, homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 24 - protein, 424 aa, homo sapiens
MSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 25 - DNA, 37 bp, Artificial
  primer for human paxillin
accttgaattcatggacgacctcgacgccctgctggc

SEQ ID NO: 26 - DNA, 39 bp, Artificial
  primer for human paxillin
ctaagcggccgcttactagcagaagagcttgaggaagca

(19) United States
(12) Patent Application Publication
(10) Pub. No.: US 2013/0136753 A1
(43) Pub. Date: May 30, 2013

(54) PAXILLIN AS A THERAPEUTIC OR DIAGNOSTIC MARKER FOR CANCER

(71) Applicants: Stephen R. Hammes, Rochester, NY (US); Aritro Sen, Rochester, NY (US)

(72) Inventors: Stephen R. Hammes, Rochester, NY (US); Aritro Sen, Rochester, NY (US)

(73) Assignee: UNIVERSITY OF ROCHESTER, Rochester, NY (US)

(21) Appl. No.: 13/805,298

(22) PCT Filed: Jun. 20, 2011
(86) PCT No.: PCT/US11/41099

§ 371 Date: Jan. 14, 2013

Related U.S. Application Data
(60) Provisional application No. 61/356,357, filed on Jun. 18, 2010.

(51) Int. Cl. G01N 33/68; A61K 31/7088

(52) U.S. Cl. CPC G01N 33/6872; A61K 31/7088
USPC 424/172.1; 436/501; 514/44 A; 536/24.5; 530/391.7; 435/7.1

(57) ABSTRACT

The present invention relates to methods for assessing the aggressiveness or proliferative activity of a cancer that is capable of both androgen-dependent and steroid-independent growth and proliferation; as well as methods and therapeutic agents for the treatment of such cancers including, among others, prostate cancers, testicular cancers, breast cancers, endometrial cancers, uterine cancers, and ovarian cancers.

This application claims the benefit of U.S. Provisional Patent Application Ser. No. 61/356,357, filed Jun. 18, 2010, which is hereby incorporated by reference in its entirety.

This invention was made with government support under grant number DK059913 awarded by the National Institutes of Health. The government has certain rights in this invention.

### FIELD OF THE INVENTION

This invention relates to the use of paxillin as a therapeutic target or diagnostic marker for cancers, including prostate cancer.

### BACKGROUND OF THE INVENTION

Prostate cancer is a major cause of morbidity and mortality in men.

Despite tremendous efforts by talented physicians and scientists, the methods of detection and treatment options for prostate cancer are still in need of improvement. There are many reasons for this slow progress, one of which is that the phenotype of prostate cancer changes dramatically with time. In the early stages of prostate cancer development, tumors are usually very dependent on androgen signaling via the androgen receptor. As such, androgen deprivation can have profound effects on tumor progression, often shrinking cancers to nearly undetectable levels. Unfortunately, within 1-2 years, prostate cancers frequently return and are then insensitive to androgen ablation therapy (castration resistant). These recurrent tumors are often deemed “androgen-independent.” However, this term is misleading since, although these tumors seem to grow in the absence of significant plasma androgen levels, in most cases they still contain functional androgen receptors and in fact still require androgen receptor signaling for growth. Why androgen receptor expression is still necessary for prostate cancer growth in the apparent absence of significant ligands remains a mystery, though several theories have been proposed, including: (1) androgen receptor or co-activator expression is amplified to increase sensitivity to remaining low levels of androgens; (2) alterations in androgen receptor sequences or co-activator populations occur to increase promiscuity of the androgen receptor to include non- or weak-androgens; (3) intracrine androgen production from within the prostate drives androgen receptor signaling; or (4) with time growth factors such as EGF or IGF indirectly activate androgen receptors in an androgen-independent fashion.

Interestingly, recent data suggest that the latter possibility, growth-factor mediated activation of the steroid receptor in the absence of steroid, may be very important. For example, in Sertoli cells and prostate cancer cell lines, androgen stimulation of extra-nuclear androgen receptors has been reported to rapidly transactivate the EGF receptor and downstream Akt and Erk kinase signaling. Similar observations have been seen in breast cancer cells, where approximately 5% of classical estrogen receptors are palmitoylated and localized at the plasma membrane via interactions with caeolin-1. Stimulation of these membrane-bound estrogen receptors can lead to EGF receptor transactivation, kinase activation, and proliferation. Similar results have been identified in numerous types of cancer, including squamuos carcinoma (Ma et al, “Role of Nongenomic Activation of Phosphatidylinositol 3-Kinase/Akt and Mitogen-Activated Protein Kinase/Extracellular Signal-Regulated Kinase Kinase/Extracellular Signal-Regulated Kinase ½ Pathways in 1,25D3-Mediated Apoptosis in Squamous Cell Carcinoma Cells,” Cancer Research 15:8131-8 (2006), thyroid cancer (Davis et al., “Overlapping Nongenomic and Genomic Actions of Thyroid Hormone and Steroids,” Steroids doi:10.1016/j.steroids.2011.02.012 (February 2011), and endometrial carcinoma (Zhang et al., “Nongenomic Effect of Estrogen on the MAPK Signaling Pathway and Calcium Influx in Endometrial Carcinoma Cells,” J. Cell Biochem 106:553-62 (2009), among others (see generally Hammes and Levin, “Extranuclear Steroid Receptors: Nature and Actions,” Endocr. Rev. 28(7):726-741 (2007)).

Physiological functions of steroids are mediated via either nuclear (genomic) or extra-nuclear (nongenomic) actions of steroid receptors. Genomic actions involve binding of steroids to steroid receptors, which then translocate to the nucleus, bind to steroid-response elements, and alter gene expression. In contrast, steroid receptors also induce rapid nongenomic signals that are generally mediated by cross-talk between the steroid receptor and either G-proteins or growth factor receptors (Lange et al., “Integration of Rapid Signaling Events with Steroid Hormone Receptor Action in Breast and Prostate Cancer,” Annu. Rev. Physiol. 69:171-199 (2007); Hammes and Levin, “Extranuclear Steroid Receptors: Nature and Actions,” Endocr. Rev. 28(7):726-741 (2007); Migliaccio et al., “Crosstalk Between EGFR and Extranuclear Steroid Receptors,” Ann. N.Y. Acad. Sci. 1089:194-200 (2006). Although transcriptional effects of steroids have been extensively studied, mechanisms regulating nongenomic actions of steroids are poorly understood. Researchers have known for decades that signaling events in the cytoplasm can have profound effects on signaling events in the nucleus; however details regarding how this cytoplasm to nuclear (“outside-inside”) cross talk is regulated are still not known.

It would be desirable, therefore, to identify an agent that can interrupt one or more regulators of non-genomic steroid actions, and thereby afford a treatment for various cancers that can develop steroid-independence such as prostate cancer and breast cancer, among others.

The present invention is directed to overcoming these and other deficiencies in the art.

### SUMMARY OF THE INVENTION

A first aspect of the present invention relates to a method of assessing aggressiveness or proliferative activity of a cancer that is capable of both steroid-dependent and steroid-independent growth and proliferation. The method includes the steps of obtaining a cancer sample from a patient; and determining whether cancer cells in the sample display an increase in the expression of paxillin or an increase in paxillin serine-phosphorylation in comparison to a control.

A second aspect of the present invention relates to a method of treating cancer that includes administering to a patient having cancer an amount of an agent that inhibits paxillin expression or activity of serine-phosphorylated paxillin, whereby said administering is effective to treat the cancer. The method is particularly useful for treating cancers that are capable, over the course of time, of both steroid-dependent and steroid-independent growth and proliferation.

A third aspect of the invention relates to a therapeutic agent that includes a first molecule that inhibits paxillin expression or activity of serine-phosphorylated paxillin, which first molecule is linked directly or indirectly to a second molecule that binds specifically to a cell surface marker of a cancer cell. Pharmaceutical compositions containing the therapeutic agent are also disclosed herein.

The accompanying examples demonstrate that paxillin, a molecule best known for regulating cytoskeletal remodeling, is a critical liaison between extranuclear and intranuclear signaling in prostate cancer cells. Previous research has shown that paxillin, a multidomain adaptor protein, is a critical regulator of testosterone-induced MAPK signaling during Xenopus oocyte maturation (Rasar et al., “Paxillin Regulates Steroid-Triggered Meiotic Resumption in Oocytes by Enhancing an All-Or-None Positive Feedback Kinase Loop,” J. Biol. Chem. 281(51):39455-39464 (2006), which is hereby incorporated by reference in its entirety). Specifically, it was found that paxillin is necessary for extranuclear Erk activation in response to multiple inputs, including nongenomic androgen signaling via membrane-localized androgen receptors (ARs) as well as growth factors via Receptor Tyrosine Kinases (RTKs). Once activated, Erk then phosphorylates paxillin on serine residues, at which point phosphoserine-paxillin travels to the nucleus and mediates AR- and Erk-dependent transcription. As demonstrated herein, if this Erk-mediated phosphorylation of paxillin is prevented, then paxillin will not enter the nucleus, androgen and growth factor-dependent transcription is abrogated, and prostate cancer cells do not proliferate in response to any of these agonists. It was shown in the examples that in androgen-dependent LnCAP cells, DHT functions as a growth factor that indirectly activates the EGF-receptor via androgen receptor binding and matrix metalloproteinase-mediated release of EGFR ligands. Interestingly, siRNA-mediated knockdown of paxillin expression in androgen-dependent LnCAP cells as well as in androgen-independent PC3 cells abrogates DHT- and/or EGF-induced Erk signaling. Furthermore, EGFR-induced Erk activation requires Src-mediated phosphorylation of paxillin on tyrosines 31/118. In contrast, paxillin is not required for PKC-induced Erk signaling. However, Erk-mediated phosphorylation of paxillin on serines 83/126/130 is still needed for both EGFR and PKC-mediated cellular proliferation. Thus, paxillin serves not only as a specific upstream regulator of Erk in response to receptor-tyrosine kinase signaling, but also as a general regulator of down-stream Erk actions regardless of agonist. Importantly, Erk-mediated serine phosphorylation of paxillin is also required for DHT-induced prostate-specific antigen mRNA expression in LnCAP cells as well as EGF-induced cyclin D1 mRNA expression in PC3 cells, indicating that paxillin regulates prostate cancer proliferation by serving as a liaison between extranuclear kinase signaling and intranuclear transcriptional signals. Both cytoplasmic paxillin and nuclear phosphoserine-paxillin expression are upregulated in human prostate cancer relative to normal prostate tissue, implying that paxillin is overactive in these tumors. Together, these data demonstrate that paxillin is a key mediator of prostate cancer growth and therefore a viable diagnostic and therapeutic target. Furthermore, since paxillin actually regulates Erk activation and downstream effects in every tumor cell line tested, regardless of origin, it is believed that paxillin is a general regulator of Erk actions well beyond the prostate.

### BRIEF DESCRIPTION OF THE DRAWINGS

FIG. 1 is a schematic illustration of a chimeric RNA molecule that includes (i) a cancer cell-specific surface antigen-binding RNA aptamer; and (ii) an RNAi molecule that inhibits expression of paxillin. The chimeric RNA molecule can be used as a therapeutic agent to inhibit paxillin expression in antigen-presenting cancer cells.

FIG. 2 is a schematic illustration of a conjugated aptamer-RNAi molecule that includes streptavidin, one or more biotin-conjugated, cancer cell-specific surface antigen-binding RNA aptamers, and one or more biotin-conjugated RNAi molecules that inhibit expression of paxillin. The conjugated aptamer-RNAi molecule can be used as a therapeutic agent to inhibit paxillin expression in antigen-expressing cancer cells.

FIG. 3 is a schematic illustration of a polycation-RNAi vector conjugated to an antibody that binds specifically to a cancer cell-specific surface antigen. The polycation-RNAi vector-antibody conjugate can be used as a therapeutic agent to inhibit paxillin expression in antigen-expressing cancer cells.

FIGS. 4A-E illustrate that DHT-induced Erk1/2 signaling occurs via MMP-mediated transactivation of EGF receptor and is regulated by paxillin. In FIG. 4A, serum-starved LnCAP cells were preincubated with vehicle (0.1% DMSO), 100 nM flutamide (androgen receptor inhibitor), 20 μM AG1478 (EGF receptor inhibitor), 5 μM Erlotinib (EGF receptor inhibitor), 20 μM PP2 (Src inhibitor), or 20 μM galardin (MMP inhibitor) for 30 min before stimulation with ethanol (Media) or 25 nM DHT for 30 minutes. Western blots of whole-cell extracts were performed for total and phosphorylated Erk1/2 (tERK½, pERK½). In FIG. 4B, A431 cells cultured in DMEM/F-12 (1:1) medium (Invitrogen) containing 10% FBS and 1% penicillin-strepto-mycin were serum-starved overnight and then stimulated with medium from DHT (25 nM), DHT (25 nM)+galardin (20 μM), or vehicle (0.1% ethanol)-treated LnCAP cells for 60 minutes. As controls, A431 cells were stimulated with DHT (25 nM) or media alone (no cells). Thereafter, A431 cells were isolated for Western blot analysis to detect phosphorylated and total EGF receptor (pEGFR, tEGFR). In FIG. 4C, serum-starved LnCAP cells were preincubated with vehicle (0.1% DMSO), 20 μM AG1478 (EGF receptor inhibitor), or 20 μM PP2 (Src inhibitor) for 30 minutes before stimulation with ethanol (Media) or 25 nM DHT for 30 minutes. Western blots of whole-cell extracts were performed for Src (tSrc, pSrc) or EGFR (tEGFR, pEGFR). In FIGS. 4D-E, LnCAP and PC3 cells were treated with non-targeting (Nsp) or paxillin-specific (Pax) siRNAs for 72 hours followed by stimulation with 25 nM DHT (4D) or 20 ng/ml EGF (4E) for the indicated times. Western blots were performed for total and phosphorylated Erk1/2, Akt (tAkt and pAkt) or total paxillin (Pax). All experiments were performed at least three times with identical results.

FIGS. 5A-D illustrate that paxillin functions upstream of Raf/MEK but downstream of the EGFR. In FIG. 5A, non-targeting (Nsp) or paxillin (Pax)-specific siRNA-treated LnCAP cells were serum-starved and then stimulated with DHT (25 nM) for the indicated times. Western blots were performed for phosphorylated and total MEK½. Cell lysates were from the same experiments represented in FIG. 1C. In FIGS. 5B-C, paxillin- or non-targeting siRNA-treated LnCAP cells were co-transfected with cDNAs encoding caMEK or caRaf for 72 hours. Serum-starved cells were then treated with 25 nM DHT for 30 minutes. Western blots detected total and phosphorylated Erk1/2 as well as paxillin. In FIG. 5D, paxillin or non-targeting siRNA-treated LnCAP cells were stimulated with DHT (25 nM) or EGF (20 ng/ml) for 30 minutes and phosphorylated, and total EGF receptor was detected by Western blot. Cell lysates were from the same experiment in FIG. 4C-D. Each experiment was performed at least three times with similar results.

FIGS. 6A-D show that paxillin is required for DHT- or EGF-induced proliferation, migration, and invasion. LnCAP and PC3 cells were treated with non-targeting (Nsp) or paxillin-specific (Pax) siRNAs for 72 hours, serum-starved overnight, and stimulated with ethanol (AA DHT (25 nM) or EGF (20 ng/ml) for 24 hours. Proliferation was assayed by 3-(4,5-dimethylthiazol-2-yl)-2,5-diphenyltetrazolium bromide (“MTT”) assay (FIG. 6A). FIG. 6B demonstrates a quantitative analysis of migration and invasion by absorbance after staining invading cells (upper panel) as well as images representing the underside of the extracellular matrix membrane containing migrated and invaded cells (lower panel). Cell migration-invasion was detected with a colorimetric QCM Cell invasion assay kit. Data are represented as the means±S.E. (n=3). *, Student's t test; p<0.05 Nsp versus Pax siRNA. In FIG. 6C, paxillin phosphorylation is shown. LnCAP cells were preincubated with vehicle (0.1% DMSO), 20 μM PP2 (Src inhibitor), or 20 μM U0126 (Erk1/2 inhibitor) for 30 minutes before stimulation with 0.1% ethanol or 25 nIV1DHT for 30 minutes. Western blots were performed for total and phosphorylated Erk1/2, total paxillin (Pax), phosphorylated paxillin at tyrosine-118 (Pax p-Y118), tyrosine-31 (Pax p-Y31), serine-83 (Pax p-S83), and serine-126 (Pax p-S126). All experiments were performed at least three times with similar results. In FIG. 6D, a model of paxillin phosphorylation is shown. DHT or EGF via indirect or direct activation of the EGFR promotes Src-mediated phosphorylation of paxillin at tyrosines 31/118, leading to activation of Raf, MEK, and Erk1/2. Activated Erk1/2 in turn regulates phosphorylation of paxillin at serines 83/126.

FIGS. 7A-B illustrate that phosphorylation of paxillin at tyrosines 31/118 and serines 83/126/130 is essential for proliferation. PC3 cells were initially transfected with Paxillin (Pax) or non-targeting (Nsp) siRNA. After 96 hours, media was removed, and cells were then either mock-transfected or transfected with wild-type paxillin (left panel) or paxillin mutated at tyrosines 31/118 (Y31A/Y118A, middle panel) or serines 83/126/130 (S83A/S126A/S130A, right panel). After 48 hours, cells were treated overnight with serum-free, phenol red-free RPMI 1640 media and stimulated with media (M) or 20 ng/ml EGF for 30 minutes (for Western blot) or 24 hours (for MTT assay). Western blots (FIG. 7A) detected total and phosphorylated Erk1/2, total paxillin (Pax), phosphorylated paxillin at tyrosine-118 (Paxp-Y118), and serine-126 (Pax p-S126). Proliferation was detected by MTT assay (FIG. 7B). Data are represented as the mean±S.E. (n=3). *, Student's t test; p≦0.05, rescue versus mock. Each experiment was performed at least three times with similar results.

FIGS. 8A-D illustrate that PMA-mediated Erk1/2 activation is paxillin-independent, but PMA-mediated proliferation still requires serine phosphorylation of paxillin. In FIG. 8A, paxillin (Pax) or non-targeting (Nsp) siRNA-treated LnCAP cells were serum-starved and treated with PMA (100 nM) or EGF (20 ng/ml) for 30 minutes. Western blots were performed for paxillin and total and phosphorylated Erk1/2. In FIG. 8B, serum-starved PC3 cells were pretreated with 0.1% DMSO or 20 μM U0126 (Erk inhibitor) for 30 minutes before stimulation with PMA (100 nM) or EGF (20 ng/ml) for another 30 minutes. Thereafter, levels of paxillin and phosphorylated paxillin at tyrosine-118 (Pax p-Y118) and serine-126 (Pax p-S126) were detected by Western blot. In FIG. 8C, cell proliferation (MTT assay) of PC3 cells treated (24 hours) with 0.1% DMSO (M) or 100 nM PMA in the presence or absence of 20 μM U0126. In FIG. 8D, PC3 cells were initially transfected with Paxillin (Pax) or non-targeting (Nsp) siRNA. After 96 hours, media were removed, and cells were then either mock-transfected or transfected with wild-type paxillin (left panel) or paxillin mutated at tyrosines 31/118 (Y31A/Y118A, middle panel) or serines 83/126/130 (S83A/S126A/S130A, right panel). After 48 hours, cells were treated overnight with serum-free, phenol red-free RPMI 1640 media and stimulated with media containing 0.1% DMSO (M) or 100 nM PMA for 24 hours. Cell proliferation was detected by MTT. Data are represented as the mean±S.E. (n=3). * Student's t test; p≦0.05 PMA versus mock. Each experiment was performed at least three times with similar results.

FIGS. 9A-C show that paxillin regulates DHT or EGF-induced Erk-mediated gene expression in prostate cancer cells. In FIG. 9A, relative expression of PSA mRNA in LnCAP cells preincubated with vehicle (0.1% DMSO), 20 μM U0126 (Erk inhibitor), or 20 μM AG1478 (EGF receptor inhibitor) for 30 minutes or treated with paxillin (Pax)-specific siRNA for 72 h before stimulation with either ethanol (M) or 25 nM DHT for 24 hours. In FIG. 9B, relative expression of cyclin D1 mRNA in PC3 cells treated with nonspecific (Nsp)- or paxillin (Pax)-specific siRNA for 96 hours followed by transfection with WT or mutated paxillin (S83A/S126A/S130A) before stimulation with either media or 20 ng/ml EGF for 24 hours. All data are represented as the mean±S.E. (n=3) and normalized to GAPDH levels. *, Student's t test; p≦0.05, stimulus versus media. All experiments were performed at least three times with similar results. In FIG. 9C, a proposed model is shown describing the paxillin role in nongenomic androgen receptor or EGF receptor signaling in prostate cancer cells. Erk mediated serine-phosphorylation of paxillin is believed to be the event immediately preceding nuclear translocation of serine-phosphorylated paxillin, which activates the cell for proliferation. Therefore, detection of serine-phosphorylated paxillin within the nuclear fraction can be used to identify those cells as cancerous.

FIG. 10 illustrates that the ability of the MMP inhibitor Galardin to block DHT-induced Erk1/2 phosphorylation can be rescued by EGF treatment. LnCAP cells cultured in RPMI-1640 medium (Invitrogen) containing 10% FBS and 1% penicillin-streptomycin were treated overnight with serum-free, phenol red-free RPMI-1640 media. Thereafter cells were treated with vehicle (0.1% DMSO) or 20 μM Galardin (MMP inhibitor, Calbiochem) for 30 minutes prior to stimulation with 0.1% ethanol (vehicle), DHT (25 nM), DHT (25 nM)+EGF (20 ng/ml), or EGF (20 ng/ml) for 30 minutes. Western blots were performed for total and phosphorylated Erk1/2.

FIGS. 11A-B show that paxillin is an important regulator of receptor tyrosine kinase-induced Erk1/2 signaling. In FIG. 11A, HEK-293 cells were treated with non-targeting (Nsp) or paxillin-specific (Pax) siRNAs for 72 hours followed by stimulation with 20 ng/ml EGF or 25 ng/ml FGF for the indicated times. Western blots were performed for total and phosphorylated Erk1/2 or total paxillin (Pax). In FIG. 11B, mouse embryonic fibroblasts from paxillin null mice were stimulated with 20 ng/ml EGF for 30 minutes followed by Western blots to detect total and phosphorylated Erk1/2 or total paxillin (Pax). All experiments were performed at least three times with identical results.

FIGS. 12A-C illustrate dose responses for EGFR inhibitors. In FIGS. 12A-B, LnCAP cells were treated overnight with serum-free, phenol red-free RPMI-1640 media. Thereafter, cells were treated with vehicle (0.1% DMSO), AG1478 or Erlotinib as indicated for 30 minutes prior to stimulation with DHT (25 nM) for 30 minutes. In FIG. 12C, AG1478 does not block FGF-induced Erk activation. LnCAP cells after overnight serum starvation were treated with or without 20 μM of AG1478 for 30 minutes followed by DHT (25 nM), EGF (20 ng/ml), or FGF (25 ng/ml).

FIGS. 13A-B illustrate that paxillin and phospho-serine paxillin are upregulated in in vivo human prostate cancer cells, but not normal prostate cells, as detected by Western (FIG. 13A) and immunohistochemistry (FIG. 13B). Total paxillin, left; phospho-serine paxillin, right.

FIG. 14 shows that androgen and EGF drive phospho-serine paxillin (PS-Pax) to the nucleus. Total paxillin (T-Pax) is primarily cytoplasmic but becomes partly nuclear with DHT and EGF. PS-Pax is almost all nuclear.

FIG. 15 illustrates DHT-induced stimulation of the PSA promoter region requires nongenomic AR signaling though Erk (U0126) and EGFR (AG1478).

FIG. 16 shows that DHT-induced stimulation of the PSA promoter requires phosphoserine-paxillin. Removal of Erk targets (S→A) eliminates PSA promoter activity in response to DHT.

FIGS. 17A-F illustrate that DHT-treatment promotes AR nuclear localization (FIG. 17B) that requires paxillin (FIG. 17C). WT paxillin rescues nuclear localization (FIG. 17E) but not mutant (Ser→Ala) paxillin that cannot be phosphorylated by Erk (FIG. 17F). Inhibition of nuclear export with Leptomycin B permits DHT-induced nuclear localization in the absence of paxillin (FIG. 17D). Thus, phospho-serine paxillin is required to retain AR in the nucleus upon DHT-stimulation. AR=androgen receptors; DAPI=4′,6-diamidino-2-phenylindole.

FIG. 18 shows that EGF stimulation of the cyclin D1 promoter requires phosphoserine-paxillin. Removal of Erk targets (S→A) eliminates cyclin D1 promoter activity in response to EGF.

### DETAILED DESCRIPTION OF THE INVENTION

One aspect of the present invention relates to a method of assessing aggressiveness or proliferative activity of a cancer that is capable of both steroid-dependent and steroid-independent growth and proliferation. The method comprises obtaining a cancer sample from a patient and determining whether cancer cells in the sample display an increase in the expression of paxillin or an increase in paxillin serine-phosphorylation in comparison to a control.

As used herein, a cancer that is capable of both steroid-dependent and steroid-independent growth and proliferation refers to types of cancers that exhibit both phases of growth and proliferation at various times during progression of the tumor. As used herein, the term “steroid-independent” refers to a cancer cell that can survive (i.e. remain alive) as well as proliferate in the absence of steroids (for example, molecules that bind to a wild-type or mutated steroid receptor; more specifically, for example, androgen, progesterone, glucocorticoid, and estrogen). The term “steroid-dependent” therefore refers to a cancer cell that requires steroid-mediated signaling via steroid receptors to remain alive. Without being limited thereto, steroid-dependent growth and proliferation of cancerous tumors often occurs during early stages of the disease whereas steroid-independent growth and proliferation of cancerous tumors often occurs during a recurrence or during late-stages of the disease. Examples of cancers that are capable of both steroid-dependent and steroid-independent growth and proliferation include, without limitation, prostate cancer, testicular cancer, breast cancer, endometrial cancer, uterine cancer, and ovarian cancer.

As used herein, the term “sample” includes but is not limited to biological fluids such as blood, serum, plasma, tissue biopsies, fractionated cells samples, and extractions. In some cases, cancer cells can be evaluated including, without limitation, cancer cells in from whole tissue (e.g., prostate, breast, or ovarian biopsies) and metastatic cancer cells in blood, urine, cellular fragments, or in tissues other than the source tissue (e.g., lung tissue and lymph node tissue), cell extracts, cell culture fluid, tissue extracts, variants thereof, or combinations thereof. In some cases, cancers cells can be evaluated to determine whether or not the cells have a cancer cell profile, including expression of certain cell surface markers.

The term “patient” in the context of the present invention refers to any living or non-living vertebrate, preferably a living or non-living mammal like a human or non-human mammal, preferably a living mammal and most preferably a living human. Whereas prostate and testicular cancer can be identified in a patient that is a male patient and ovarian, uterine, and endometrial cancer can be identified in a female patient, breast cancer can be identified in either a male patient or a female patient.

Any method can be used to obtain a sample from an individual. For example, a blood sample can be obtained by peripheral venipuncture, and urine samples can be obtained using standard urine collection techniques. In some cases, a tissue sample can be obtained from a tissue biopsy (e.g., a needle biopsy), from a resection of the cancerous tissue, or from removal of the entire affected tissue (e.g., a radical prostatectomy, ovariectomy, or mastecomy). A sample can be manipulated prior to being evaluated. A sample also can be manipulated prior to being evaluated for a cancer fluid profile or a cancer cell profile. For example, a biopsy specimen can be frozen, embedded, and/or sectioned prior to being evaluated. In addition, paxillin can be extracted from a sample, purified, if desired, and evaluated to determine the level of paxillin expression or the nuclear or total levels of serine-phosphorylated paxillin. In some cases, a tissue sample can be disrupted to obtain a cell lysate or a nuclear lysate, which can then be evaluated. In some cases, individual cells can be isolated from the sample or separated from other cells or tissues prior to analysis. For example, cancer cells can be isolated from normal tissues using laser capture microdissection and then the isolated cancer cells or both the cancer cells and normal cells can be evaluated for their paxillin expression or the nuclear or total level of serine-phosphorylated paxillin.

Any method can be used to determine cytoplasmic levels of paxillin or nuclear or total levels of serine-phosphorylated paxillin relative to corresponding control samples. For example, the increase in the expression of paxillin or the increase in paxillin phosphorylation can be measured using, without limitation, immuno-based assays (e.g., ELISA, Western blotting, and immunohistochemistry), arrays for detecting polypeptides, two-dimensional gel analysis, chromatographic separation, mass spectrometry (MS), tandem mass spectrometry (MS/MS), or liquid chromatography (LC)-MS. Detection of paxillin using immunoassays can be carried out using commercially available anti-paxillin antibodies or anti-phosphoserine paxillin antibodies, as well as binding fragments thereof. Anti-phosphoserine paxillin antibodies suitable to detect nuclear levels of phosphoserine paxillin include, without limitation, polyclonal or monoclonal antibodies, and binding fragments thereof, that react with phosphorylated residues Ser-83, Ser-126 and/or Ser-130. Ser-126 and Ser-130 are often phosphorylated contemporaneously. Exemplary antibodies for detection of phospho-serine paxillin include, without limitation, paxillin phospho-Ser126 (Invitrogen) and paxillin phospho-Ser83 (ECM Biosciences).

Methods provided herein for identifying cancer in patients can be used in combination with one or more methods typically used to identify prostate cancer, testicular cancer, breast cancer, uterine cancer, endometrial cancer, or ovarian cancer. For example, methods for the diagnosis of prostate cancer include, without limitation, digital rectal exam, transrectal ultrasonography, intravenous pyelogram, cystoscopy, and blood and urine tests for levels of prostatic acid phosphatase (PAP) and PSA. Exemplary methods for the diagnosis and monitoring of breast cancer include manual self-examination or examination by a professional, mammogram, magnetic resonance imaging, and blood tests for levels of the certain markers (e.g., CA 15.3 for breast and ovarian cancers, CA 27.29 for breast cancer, and CAl25 for breast cancer recurrence or ovarian cancer), and CT scans. Exemplary methods for the diagnosis and monitoring of ovarian cancer include ultrasound, magnetic resonance imaging, CT scans, and blood tests for certain markers (including those listed above). A patient can be evaluated regularly for these and others cancer. For example, a patient can be evaluated once a year for life. In some cases, male humans can be evaluated for prostate cancer once every year beginning at age 35; and female patients can be evaluated for breast or ovarian cancer once every year beginning at age 35. Patients that are susceptible to develop these and other cancers can be screened more frequently, and screening can be started at an earlier age. For example, individuals having a genetic predisposition to develop cancer, a family history of cancer, or prior diagnosis of cancer can be screened more frequently.

Methods typically used to assess the aggressiveness of prostate cancer in a patient include determining the Gleason score, the serum PSA level, and whether or not the serum PSA level increases over time as well as rate of PSA increases (PSA velocity). The Gleason score is a measure of how different cancer cells are from normal cells. The more different the cancer cells are from non-cancer cells, the more likely that the cancer will spread quickly.

The greater the expression level of paxillin or paxillin serine-phosphorylation in a patient sample, the more aggressive and proliferative the cancer is likely to be. In certain embodiments, the increase in the expression of paxillin is measured by detecting the level of paxillin transcripts, the level of paxillin in the cytoplasm, the total level of paxillin serine-phosphorylation, or the level of serine-phosphorylated paxillin in the nucleus. In certain instances, both the level of paxillin in the cytoplasm and the level of nuclear serine-phosphorylated paxillin are measured. The greater the differences between the paxillin expression levels or paxillin serine-phosphorylation levels in a cancer sample from a patient and the corresponding levels in a control sample or a range of values considered normal for non-cancerous cells, then the more likely the cancer will be aggressive, exhibiting strong proliferation and motility. In some cases, the levels of paxillin or paxillin serine-phosphorylation can be used in combination with one or more other factors to determine whether or not a patient having a particular form of cancer is susceptible to a poor outcome. For example, levels of paxillin or paxillin serine-phosphorylation a prostate cancer sample can be used in combination with the clinical stage, the serum PSA level, and/or the Gleason pattern of the prostate cancer to determine whether or not the patient is likely to have to a poor outcome. Similar analyses can be performed using criteria suitable for breast cancer, ovarian cancer, and the like, including tumor size and marker expression levels, e.g., brc-1, her2, her3, etc.

In one embodiment, the control cell is a normal prostate cell and the corresponding expression of paxillin or the level of paxillin serine-phosphorylation in the normal prostate cell (or its nucleus). Similarly, the control cell can also be a normal ovarian, testicular, uterine, breast, or endometrial cell, and the corresponding expression of paxillin or the level of paxillin serine-phosphorylation in the normal cell (or its nucleus). In another embodiment, the control cell is a non-aggressive cancer cell and the expression of paxillin or the level of paxillin serine-phosphorylation is that of the non-aggressive cancer cell.

Information about the aggressiveness of the cancer can be used to guide treatment selection. For example, an individual identified as having more aggressive form of cancer can be treated earlier and more aggressively than an individual identified as having less aggressive cancer. A more aggressive treatment can include, for example, radical prostatectomy, ovariectomy, or mastectomy alone or in combination with one or more chemo-, radio-, or immunotherapies. An individual identified as having less aggressive form of cancer may undergo “watchful waiting” while having little or no standard treatment, particularly if the individual is elderly.

Once cancer has been identified in an individual, the individual can be subsequently evaluated or monitored over time for progression of the cancer, particularly if the cancer was identified as being aggressive. In one embodiment, the monitoring may include obtaining a second sample from a patient, and determining whether cancer cells in the second sample display an increase in the expression of paxillin or an increase in the level of paxillin serine-phosphorylation in comparison to a control and/or in comparison to the first sample. For example, the cancer in an individual can be assessed as having progressed if it is determined that the later-obtained sample contains a level of paxillin or serine-phosphorylated paxillin that is greater than the level observed in a corresponding sample obtained previously from the same individual.

An individual can be monitored for progression of cancer over any period of time, and with any frequency of testing. For example, the individual can be monitored once a year, twice a year, three times a year, or more frequently. In some cases, an individual can be monitored every three months for five years or once a year for as long as the individual is alive. In one embodiment, the obtaining of a second cancer sample occurs following a delay of at least 7, 14, or 21 days following obtaining the previous cancer sample. In yet a further embodiment, the obtaining of a second cancer sample occurs following administration of a treatment protocol to the patient, and preferably at least 7, 14, or 21 days following the last administration of the treatment protocol.

An individual can also be assessed for progression of cancer before, during, and after treatment. For example, an individual can be assessed for progression (e.g., metastasis) of cancer while being treated with traditional chemo-, radio-, or immunotherapies, or following surgery to remove the primary tumor. Assessing an individual for progression of cancer during treatment can allow the effectiveness of the cancer therapy to be determined. For example, a decrease in the level of paxillin or paxillin serine-phosphorylation in a sample from an individual being treated can be compared to the paxillin or paxillin serine-phosphorylation level observed in a corresponding sample obtained previously from the same individual, thereby indicating that the therapy is effective. In some instances, a therapy can be assessed as being effective if it is determined that the paxillin or serine-phosphorylated paxillin levels are observed to decrease following treatment.

A second aspect of the present invention relates to a method of treating cancer and therapeutic agents for treating cancer. The method of treatment includes administering to a patient having cancer an amount of an agent that inhibits paxillin expression or activity of serine-phosphorylated paxillin, whereby said administering is effective to treat the cancer. The method is particularly useful for treating cancers that are capable, over the course of time, of both steroid-dependent and steroid-independent growth and proliferation, such as prostate cancer, testicular cancer, breast cancer, ovarian cancer, uterine cancer, and endometrial cancer. Other cancers that exhibit, over the course of time, both steroid-dependent and steroid-independent growth and proliferation can also be treated. Thus, the methods of the present invention can be used to treat these cancers while the cancer proliferates in a steroid-dependent manner or while the cancer proliferates in a steroid-independent manner.

In accordance with one embodiment, the treatment of estrogen-independent breast, ovarian, endometrial, and uterine cancers that are non-responsive to anti-estrogen therapy is contemplated herein. In accordance with another embodiment, the treatment of estrogen-dependent breast, ovarian, endometrial, and uterine cancers is contemplated. In accordance with a further embodiment, the treatment of androgen-independent prostate, testicular, and breast cancers that are non-responsive to androgen blockade is contemplated herein. In addition, in yet another embodiment, the treatment of androgen-dependent prostate, testicular, and breast cancers cancers is contemplated.

According to one embodiment, the therapeutic agent that inhibits paxillin expression or activity of serine-phosphorylated paxillin includes an interfering RNA (“RNAi”) molecule, which when introduced into a targeted cancer cell inhibits paxillin expression and, thus, subsequent serine-phosphorylation of paxillin, particularly that caused by Erk. In certain embodiments, the RNAi agent is siRNA, shRNA, miRNA, or another antisense RNA molecule that disrupts stability of paxillin transcripts.

An antisense nucleic acid can be designed such that it is complementary to the entire coding region of paxillin mRNA, but more preferably is an oligonucleotide that is antisense to only a portion of the coding or noncoding region of paxillin mRNA. For example, the antisense oligonucleotide can be complementary to the region surrounding the translation start site of paxillin mRNA, e.g., between the −10 and +10 regions of the target gene nucleotide sequence. An antisense oligonucleotide can be, for example, about 15, 20, 25, 30, 35, 40, 45, 50, 55, 60, 65, 70, 75, 80, or more nucleotides in length.

An antisense or RNAi nucleic acid of the invention can be constructed using chemical synthesis and enzymatic ligation reactions with procedures known in the art. For example, an antisense or RNAi nucleic acid can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and modified nucleotides can be used. Examples of modified nucleotides which can be used to generate the modified RNAi nucleic acids include 5-fluorouracil, 5-bromouracil, 5-chlorouracil, 5-iodouracil, hypoxanthine, xanthine, 4-acetylcytosine, 5-carboxyhydroxylmethyluracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, and 2,6-diaminopurine.

An important feature of RNAi affected by siRNA is the double stranded nature of the RNA and the absence of large overhanging pieces of single stranded RNA, although dsRNA with small overhangs and with intervening loops of RNA has been shown to effect suppression of a target gene. As used herein, it will be understood that the terms siRNA and RNAi are interchangeable. Furthermore, as is well-known in the field, RNAi technology may be effected by siRNA, miRNA or shRNA or other RNAi inducing agents. Although siRNA will be referred to in general in the specification, it will be understood that any other RNA interfering agents may be used, including shRNA, miRNA or an RNAi-inducing vector whose presence within a cell results in production of an siRNA, shRNA, or miRNA targeted to a paxillin transcript.

RNA interference is a multistep process and is generally activated by double-stranded RNA (dsRNA) that is homologous in sequence to the targeted paxillin gene. Introduction of long dsRNA into the cells of organisms leads to the sequence-specific degradation of homologous gene transcripts. The long dsRNA molecules are metabolized to small (e.g., 21-23 nucleotide (nt)) interfering RNAs (siRNAs) by the action of an endogenous ribonuclease known as Dicer. The siRNA molecules bind to a protein complex, termed RNA-induced silencing complex (RISC), which contains a helicase activity and an endonuclease activity. The helicase activity unwinds the two strands of RNA molecules, allowing the antisense strand to bind to the targeted paxillin mRNA molecule. The endonuclease activity hydrolyzes the paxillin mRNA at the site where the antisense strand is bound. Therefore, RNAi is an antisense mechanism of action, as a single stranded (ssRNA) RNA molecule binds to the target paxillin mRNA molecule and recruits a ribonuclease that degrades the paxillin mRNA.

An “RNAi-inducing agent” or “RNAi molecule” is used in the invention and includes for example, siRNA, miRNA or shRNA targeted to a paxillin transcript or an RNAi-inducing vector whose presence within a cell results in production of a siRNA or shRNA targeted to the target paxillin transcript. Such siRNA or shRNA comprises a portion of RNA that is complementary to a region of the target paxillin transcript. Essentially, the “RNAi-inducing agent” or “RNAi molecule” downregulates expression of the targeted paxillin molecule via RNA interference.

Exemplary RNAi specific for knock-down of human paxillin expression levels include, without limitation, the following siRNA:

(sense, SEQ ID NO: 1)
5′-GUGUGGAGCCUUCUUUGGU-3′
and
(antisense, SEQ ID NO: 2)
5′-ACCAAAGAAGGCUCCACAC-3′;
(sense, SEQ ID NO: 3)
5′-CCACACAUACCAGGAGAUU-3′
and
(antisense, SEQ ID NO: 4)
5′-AAUCUCCUGGUAUGUGUGG-3′;
(sense, SEQ ID NO: 5)
5′-GAACGACAAGCCUUACUGU-3′
and
(antisense, SEQ ID NO: 6)
5′-ACAGUAAGGCUUGUCGUUC-3′;
(sense, SEQ ID NO: 7)
5′-CAACUGGAAACCACACAUAUU-3′
and
(antisense, SEQ ID NO: 8)
5′-UAUGUGUGGUUUCCAGUUGUU-3′;
(sense, SEQ ID NO: 9)
5′-GUCUCUUGGAUGAACUGGAUU-3′
and
(antisense, SEQ ID NO: 10);
5′-UCCAGUUCAUCCAAGAGACUU-3′;
(sense, SEQ ID NO: 11)
5′-CCCUGACGAAAGAGAAGCCUAUU-3′
and
(antisense, SEQ ID NO: 12)
5′-UAGGCUUCUCUUUCGUCAGGGUU-3′;
and
(sense, SEQ ID NO: 13)
5′-CGGGCCAUCCUGGAGAACUAUAUCU-3′
and
(antisense, SEQ ID NO: 14)
5′-AGAUAUAGUUCUCCAGGAUGGCCCG-3′.

These siRNA molecules can be modified to include dT nucleotides or other modified nucleotides as described above. Other RNAi molecules specific for knock-down of paxillin expression can be designed using the Whitehead Institute siRNA Selection Program available online at the Massachusetts Institute of Technology internet site, or the BLOCK-iT™ RNAi Designer program available online from the Invitrogen internet site. The above-identified siRNA molecules can easily be converted to shRNA using well-known protocols, including those available from the above-identified internet sites. Moreover, these siRNA sense/antisense molecules can be used to replace mir-30 sequences in the shRNAmir construct available from ThermoFisher to afford a miRNA molecule suitable for paxillin knock-down (see Silva et al., “Second-generation shRNA Libraries Covering the Human and Mouse Genomes,” Nature Genetics 37(11):1281-88 (2005), which is hereby incorporated by reference in its entirety).

Commercially available sources of paxillin-specific RNAi include, without limitation, a mixture of three specific siRNA from OriGene (product # SR303929); siRNA from Santa Cruz Biotechnology (product # sc-29439), shRNA from Santa Cruz Biotechnology (product # sc-29439-sh), and shRNA available from OriGene (product # TR316429). In the accompanying Examples, siRNA from Santa Cruz Biotechnology (product # sc-29439) was utilized.

Various delivery methods suitable for the delivery of the RNAi inducing agent (including siRNA, shRNA and miRNA, etc) may be used. For example, some delivery agents for the RNAi-inducing agents are selected from the following non-limiting group of cationic polymers, modified cationic polymers, peptide molecular transporters, lipids, liposomes and/or non-cationic polymers. Examples of such polymers include, without limitation, polyethylenimine (e.g., linear or branched PEI) and/or polyethylenimine derivatives, grafted PEIs such as galactose PEI, cholesterol PEI, antibody derivatized PEI, and polyethylene glycol PEI (PEG-PEI) derivatives thereof (see, e.g., Ogris et al., AAPA Pharm Sci 3:1-11 (2001); Furgeson et al., Bioconjugate Chem., 14:840-847 (2003); Kunath et al., Pharmaceutical Res, 19: 810-817 (2002); Choi et al., Bull. Korean Chem. Soc. 22:46-52 (2001); Bettinger et al., Bioconjugate Chem. 10:558-561 (1999); Peterson et al., Bioconjugate Chem. 13:845-854 (2002); Erbacher et al., J. Gene Medicine Preprint 1:1-18 (1999); Godbey et al., Proc Natl Acad Sci USA 96:5177-5181 (1999); Godbey et al., J Controlled Release 60:149-160 (1999); Diebold et al., J Biol Chem 274:19087-19094 (1999); Thomas and Klibanov, Proc Natl Acad Sci USA 99:14640-14645 (2002); and U.S. Pat. No. 6,586,524 to Sagara, each of which is hereby incorporated by reference in its entirety.

The siRNA molecule can also be present in the form of a bioconjugate, for example a nucleic acid conjugate as described in U.S. Pat. No. 6,528,631, U.S. Pat. No. 6,335,434, U.S. Pat. No. 6,235,886, U.S. Pat. No. 6,153,737, U.S. Pat. No. 5,214,136, or U.S. Pat. No. 5,138,045, each of which is hereby incorporated by reference in its entirety.

As a further example, yet another delivery route includes the direct delivery of RNAi inducing agents (including siRNA, shRNA and miRNA) and even anti-sense RNA (asRNA) via gene constructs followed by the transformation of cells. This results in the transcription of the gene constructs encoding the RNAi inducing agent, such as siRNA, shRNA and miRNA, or even asRNA and provides for the transient or stable expression of the RNAi inducing agent in those transformed cells. Viral vector delivery systems may also be used.

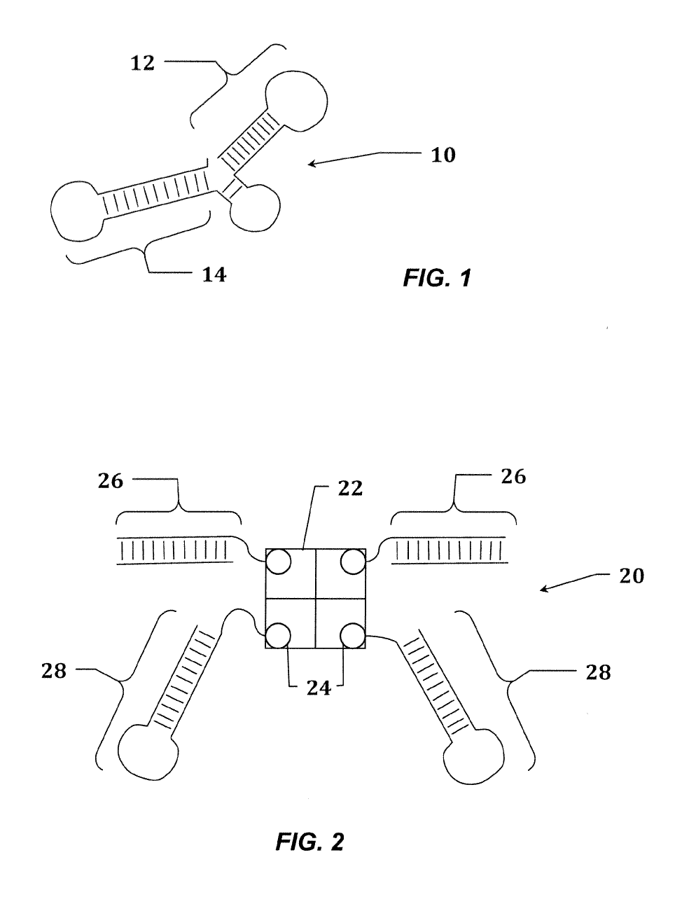

Targeted delivery strategies may also be utilized so as to deliver the RNAi directly to the cancer cells of interest. In one approach, illustrated in FIG. 1, a chimeric RNA molecule 10 is provided, which includes an RNA aptamer portion 12 that binds specifically to a cancer cell surface marker and an RNAi molecule 14 of the type described above, which is capable of knocking down expression levels of paxillin.

Aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence. Aptamers may also include a promoter or primer-annealing sequence needed to amplify, transcribe, or replicate all or part of the aptamer molecule or sequence.

Nucleic acid aptamers, as used herein, include monovalent aptamers and multivalent (including bivalent) aptamers. Methods of making bivalent and multivalent aptamers and their expression in multi-cellular organisms are described in U.S. Pat. No. 6,458,559 to Shi & L is, which is hereby incorporated by reference in its entirety. A method for modular design and construction of multivalent nucleic acid aptamers, their expression, and methods of use are described in U.S. Patent Application Publication No. 2005/0282190 to Shi et al., which is hereby incorporated by reference in its entirety.

Identifying suitable nucleic acid aptamers that bind specifically to a cancer cell surface marker with sufficiently high affinity (e.g., Kd≦50 nM) and specificity from a pool of nucleic acids containing a random region of varying or predetermined length can be carried out using the whole cell SELEX procedure (Guo et al., “CELL-SELEX: Novel Perspectives of Aptamer-Based Therapeutics,” Int J Mol Sci. 9(4): 668-678 (2008); Cerchia et al., “Neutralizing Aptamers from Whole-Cell SELEX Inhibit the RET Receptor Tyrosine Kinase,” PLoS Biol 3(4):e123 (2005), each of which is hereby incorporated by reference in its entirety). Basically, the established in vitro selection and amplification scheme known as SELEX (see U.S. Pat. No. 5,270,163 to Gold & Tuerk, Ellington & Szostak, “In Vitro Selection of RNA Molecules That Bind Specific Ligands,” Nature 346:818-22 (1990), and Tuerk & Gold, “Systematic Evolution of Ligands by Exponential Enrichment: RNA Ligands to Bacteriophage T4 DNA Polymerase,” Science 249:505-10 (1990), each of which is hereby incorporated by reference in its entirety) is performed on whole cells, in this case cancer cells, with repeated rounds of selection and counter-selection to arrive at an enriched pool of cancer cell-binding aptamers. This whole cell SELEX procedure can be modified so that an entire pool of aptamers with binding affinity can be identified by selectively partitioning the pool of aptamers as described in U.S. Patent Application Publication No. 2004/0053310 to Shi et al., which is hereby incorporated by reference in its entirety.

Using this whole cell SELEX procedure, a number of high affinity cancer cell-targeting aptamers have been identified including, without limitation, the following:
(1) Aptamer A10 (5′-GGGAGGACGAUGCGGAUCAGCCAUGUUUACGUCAC UCCUUGUCAAUCCUCAUCGGCAGACGACUCGCCCGA-3′, SEQ ID NO: 15), which binds specifically to prostate-specific membrane antigen (“PSMA”) (Lupold et al., “Identification and Characterization of Nuclease-stabilized RNA Molecules That Bind Human Prostate Cancer Cells via the Prostate-specific Membrane Antigen,” Cancer Res. 62:4029 (2002); McNamara et al., “Cell Type-Specific Delivery of siRNAs with Aptamer siRNA Chimeras,” Nature Biotech. 24(8):1005-1015 (2006), each of which is hereby incorporated by reference in its entirety);
(2) Aptamer A10-3 (5′-GGGAGGACGAUGCGGAUCAGCCAUGUUUACGUCAC UCCUUGUCAAUCCUCAUCGGC-3′, SEQ ID NO: 16), which binds specifically to PSMA (Ni et al., “Prostate-targeted Radiosensitization via Aptamer-shRNA Chimeras in Human Tumor Xenografts,” J. Clin. Invest. 121(6):2383-2390 (2011); McNamara et al., “Cell Type-Specific Delivery of siRNAs with Aptamer siRNA Chimeras,” Nature Biotech. 24(8):1005-1015 (2006), each of which is hereby incorporated by reference in its entirety);
(3) Aptamer A9 (5′-GGGAGGACGAUGCGGACCGAAAAAGACCUGACUUCUA UACUAAGUCUACGUUCCCAGACGACUCGCCCGA-3′, SEQ ID NO: 17), which binds specifically to PSMA (Lupold et al., “Identification and Characterization of Nuclease-stabilized RNA Molecules That Bind Human Prostate Cancer Cells via the Prostate-specific Membrane Antigen,” Cancer Res. 62:4029 (2002); Chu et al., “Aptamer mediated siRNA Delivery,” Nucl Acids Res. 2006(34):e73 (2006), each of which is hereby incorporated by reference in its entirety);
(4) Aptamer A30 (5′-UAAUACGACUCACUAUAGGGAAUUCCGCGUGUGCCA GCGAAAGUUGCGUAUGGGUCACAUCGCAGGCACAUGUCAUCUGGGCG GUCCGUUCGGGAUCCUC-3′, SEQ ID NO: 18), which binds specifically to the oligomeric state of the extracellular domain of human epidermal growth factor receptor-3 (HER3) which is overexpressed in breast cancer cells (Chen et al., “Inhibition of Heregulin Signaling by an Aptamer that Preferentially Binds to the Oligomeric Form of Human Epidermal Growth Factor Receptor-3,” Proc. Natl. Acad. Sci. USA 100(16):9226-9231 (2003), which is hereby incorporated by reference in its entirety);
(5) Aptamer S6 (5′-GGGAGAUACCAGCUUAUUCAAUUUGGAUGGGGAGAUC CGUUGAGUAAGCGGGCGUGUCUCUCUGCCGCCUUGCUAUGGGGAGAU AGUAAGUGCAAUCU-3′, SEQ ID NO: 19), which binds specifically to HER-2 overexpressing breast cancer cells (Kang et al., “Isolation of RNA Aptamers Targeting HER-2-overexpressing Breast Cancer Cells Using Cell-SELEX,” Bull. Korean Chem. Soc. 30(8):1827-1831 (2009), which is hereby incorporated by reference in its entirety); and
(6) Aptamer aptTOV1 (5′-UCCAGAGUGACGCAGCAGAUCUGUGUAGGAUCG CAGUGUAGUGGACAUUUGAUACGACUGGCUCGACACGGUGGCUUA-3′, SEQ ID NO: 20), which binds with high affinity to the ovarian clear cell carcinoma cell line TOV-21G (Van Simaeys et al., “Study of the Molecular Recognition of Aptamers Selected through Ovarian Cancer Cell-SELEX,” Plos One 5(11):e13770 (2010), which is hereby incorporated by reference in its entirety).

Before joining two functional RNA molecules, it is often beneficial to first predict the secondary structures of the chimeric nucleic acid molecule to ensure that their combination is unlikely to disrupt their secondary structures. Secondary structure predictions can be performed using a variety of software including, without limitation, RNA Structure Program (Dr. David Mathews, University of Rochester) and MFold (Dr. Michael Zuker, The RNA Institute, SUNY at Albany), among others. If the secondary structure predictions suggest no problems, then the chimeric nucleic acid molecules can be generated. Double-stranded DNA templates can be prepared by cloning their PCR products into a cloning vector and using the clones as templates for PCR with the appropriate primers (e.g., 5′ primer for the aptamer portion and 3′ primer for the siRNA, or vice versa depending on the orientation of the aptamer and RNAi portions). These same primers can be used to generate the chimeric DNA template for transcription, and in vitro transcription can be carried out using standard procedures to obtain the RNA chimeras, which can then be gel purified prior to use.

One embodiment of the therapeutic agent of FIG. 1 includes a PSMA-specific aptamer and a paxillin RNAi molecule, which is targeted to prostate cancer cells expressing PSMA. Upon binding of the PSMA-specific aptamer to the PSMA-expressing cancer cell, the cancer cell will take up the molecule and the RNAi molecule will interfere with paxillin expression and thereby also reduce the nuclear translocation of phospho-serine paxillin. The disruption of paxillin expression and nuclear translocation of phospho-serine paxillin will diminish both proliferation and survival of the targeted cancer cell.

Another embodiment of the therapeutic agent of FIG. 1 includes a HER2 breast cancer cell-specific aptamer and a paxillin RNAi molecule, which is targeted to breast cancer cells expressing HER2. Upon binding of the aptamer to the HER2-expressing breast cancer cell, the cancer cell will take up the molecule and the RNAi will interfere with paxillin expression and thereby also reduce the nuclear translocation of phospho-serine paxillin. The disruption of paxillin expression and nuclear translocation of phospho-serine paxillin will diminish both proliferation and survival of the targeted cancer cell.

A further embodiment of the therapeutic agent of FIG. 1 includes an aptamer that targets ovarian clear cell carcinoma cells and a paxillin RNAi molecule, which is targeted to ovarian clear cell carcinomas. Upon binding of the aptamer to the ovarian clear cell carcinoma cell, the cancer cell will take up the molecule and the RNAi will interfere with paxillin expression and thereby also reduce the nuclear translocation of phospho-serine paxillin. The disruption of paxillin expression and nuclear translocation of phospho-serine paxillin will diminish both proliferation and survival of the targeted cancer cell.

In another approach for targeted delivery of the therapeutic agent, illustrated in FIG. 2, a conjugated aptamer-RNAi molecule 20 is provided. In this embodiment, one or more RNAi molecules that inhibit expression of paxillin 26 and one or more cancer cell-specific binding aptamers 28 form the functional components of the conjugate 20. (In FIG. 2, two of each are shown.) All four of these molecules are biotinylated 24, and the conjugate is formed upon incubation of the biotinylated RNAi and aptamers with streptavidin 22. Biotinylation of the siRNA at their 5′ ends and biotinylation of the aptamers at their 3′ ends is known not to interfere with the activity of these RNA molecules (see Chu et al., “Aptamer Mediated siRNA Delivery,” Nucl Acids Res. 34(10):e73 (2006), which is hereby incorporated by reference in its entirety.

One embodiment of the therapeutic agent of FIG. 2 includes one or more biotinylated PSMA-specific aptamers and one or more biotinylated paxillin RNAi molecules, which is targeted to prostate cancer cells expressing PSMA. Upon binding of the PSMA-specific aptamer to the PSMA-expressing cancer cell, the cancer cell will take up the conjugate, the biotinylated aptamer will dissociate from streptavidin, and the RNAi molecule will interfere with paxillin expression and thereby also reduce the nuclear translocation of phospho-serine paxillin. The disruption of paxillin expression and nuclear translocation of phospho-serine paxillin will diminish both proliferation and survival of the targeted cancer cell.

Another embodiment of the therapeutic agent of FIG. 2 includes one or more biotinylated aptamers specific for a HER2 breast cancer cell and a biotinylated paxillin RNAi molecule, which is targeted to breast cancer cells expressing HER2. Upon binding of the aptamer to the HER2-expressing breast cancer cell, the cancer cell will take up the conjugate, the biotinylated aptamer will dissociate from streptavidin, and the RNAi will interfere with paxillin expression and thereby also reduce the nuclear translocation of phospho-serine paxillin. The disruption of paxillin expression and nuclear translocation of phospho-serine paxillin will diminish both proliferation and survival of the targeted cancer cell.

A further embodiment of the therapeutic agent of FIG. 2 includes one or more biotinylated aptamers that target ovarian clear cell carcinoma cells and one or more biotinylated paxillin RNAi molecules, which is targeted to ovarian clear cell carcinomas. Upon binding of the aptamer to the ovarian clear cell carcinoma cell, the cancer cell will take up the conjugate, the biotinylated aptamer will dissociate from streptavidin, and the RNAi will interfere with paxillin expression and thereby also reduce the nuclear translocation of phospho-serine paxillin. The disruption of paxillin expression and nuclear translocation of phospho-serine paxillin will diminish both proliferation and survival of the targeted cancer cell.

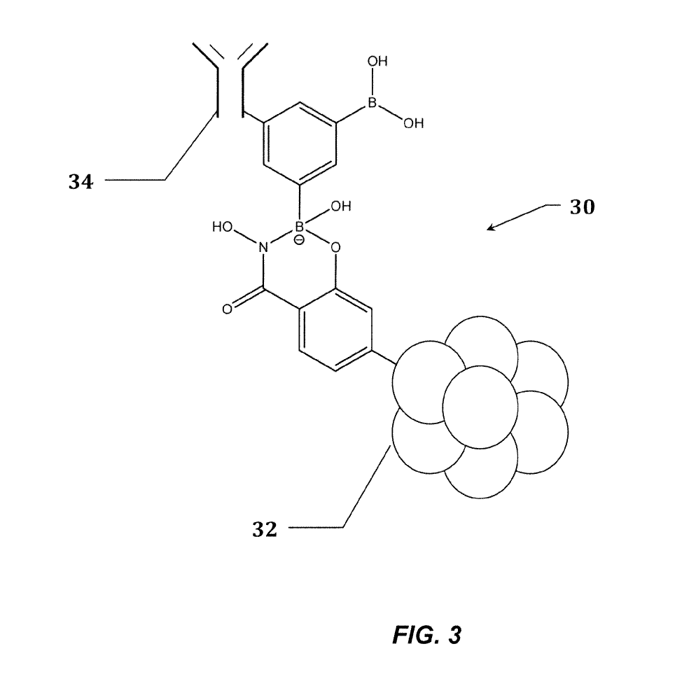

In a further approach for targeted delivery of the therapeutic agent, illustrated in FIG. 3, a conjugate 30 includes a polycation-RNAi vector 32 linked via phenyl(di)boronic acid-salicylhydroxamic acid assembly to an antibody 34 that is specific for a cancer cell surface marker. In this embodiment, the phenyl(di)boronic acid is first coupled to the antibody via a PEG linker using the methodology of Moffatt et al., “Successful in vivo Tumor Targeting of Prostate-specific Membrane Antigen with a Highly Efficient J591/PEI/DNA Molecular Conjugate,” Gene Therapy 13:761-772 (2006), which is hereby incorporated by reference in its entirety. The salicylhydroxamic acid is coupled to polyethyleneimine (PEI), a polycation, using the procedures of Moffatt et al. (“Successful in vivo Tumor Targeting of Prostate-specific Membrane Antigen with a Highly Efficient J591/PEI/DNA Molecular Conjugate,” Gene Therapy 13:761-772 (2006), which is hereby incorporated by reference in its entirety), and thereafter the RNAi can be introduced to the SHA-PEI solution to form the self-assembled conjugate 30.

One embodiment of the therapeutic agent of FIG. 3 includes a PSMA-specific antibody and one or more paxillin RNAi molecules in the PEI matrix, which are conjugated together via PDB-SHA bridge. This conjugate is targeted to prostate cancer cells expressing PSMA. Upon binding of the PSMA-specific antibody to the PSMA-expressing prostate cancer cell, the cancer cell will take up the conjugate and the RNAi molecule will interfere with paxillin expression and thereby also reduce the nuclear translocation of phospho-serine paxillin. The disruption of paxillin expression and nuclear translocation of phospho-serine paxillin will diminish both proliferation and survival of the targeted cancer cell.

Another embodiment of the therapeutic agent of FIG. 3 includes a HER2-specific monoclonal antibody (e.g., Trastuzumab (Herceptin®, Genentech)) and one or more paxillin RNAi molecules in the PEI matrix, which are conjugated together via PDB-SHA bridge. This conjugate is targeted to breast cancer cells expressing HER2. Upon binding of the HER2-specific antibody to the HER2-expressing breast cancer cell, the cancer cell will take up the conjugate and the RNAi molecule will interfere with paxillin expression and thereby also reduce the nuclear translocation of phospho-serine paxillin. The disruption of paxillin expression and nuclear translocation of phospho-serine paxillin will diminish both proliferation and survival of the targeted cancer cell.

According to another embodiment, the therapeutic agent that inhibits activity of serine-phosphorylated paxillin is a nucleic acid aptamer selected for binding to non-phosphorylated serine residues of paxillin. In certain embodiments, the aptamer recognizes non-phosphorylated serine residues 83, 126, 130, or a combination thereof. Aptamers can be raised against these residues using conventional SELEX procedure, described above, with suitable polypeptide fragments of paxillin and counter-selection against serine-phosphorylated paxillin. The capacity for binding to whole paxillin can also be screened, and optionally counter-selected during SELEX.

Paxillin polypeptides useful for can be synthesized using standard so lid-phase peptide coupling procedures or using recombinant technology. The nucleotide sequence of human paxillin is identified at Genbank Accession NM_{—}001080855, which is hereby incorporated by reference in its entirety, and shown below as SEQ ID NO: 21.

atggacgacctcgacgccctgctggcggacttggagtctaccacctcccacatctccaa
acggcctgtgttcttgtcggaggagaccccctactcatacccaactggaaaccacacat
accaggagattgccgtgccaccccccgtccccccacccccgtccagcgaggccctcaat
ggcacaatccttgaccccttagaccagtggcagcccagcagctcccgattcatccacca
gcagcctcagtcctcatcacctgtgtacggctccagtgccaaaacttccagtgtctcca
accctcaggacagtgttggctctccgtgctcccgagtgggtgaggaggagcacgtctac
agcttccccaacaagcagaaatcagctgagccttcacccaccgtaatgagcacgtccct
gggcagcaacctttctgaactcgaccgcctgctgctggaactgaacgctgtacagcata
acccgccaggcttccctgcagatgaggccaactcaagccccccgcttcctggggccctg
agccccctctatggtgtcccagagactaacagccccttgggaggcaaagctgggcccct
gacgaaagagaagcctaagcggaatgggggccggggcctggaggacgtgcggcccagtg
tggagagtctcttggatgaactggagagctccgtgcccagccccgtccctgccatcact
gtgaaccagggcgagatgagcagcccgcagcgcgtcacctccacccaacagcagacacg
catctcggcctcctctgccaccagggagctggacgagctgatggcttcgctgtcggatt
tcaagatccagggcctggagcaaagagcggatggggagcggtgctgggcggccggctgg
cctcgggacggcgggcggagcagccccggagggcaggacgagggagggttcatggccca
ggggaagacagggagcagctcaccccctggggggcccccgaagcccgggagccagctgg
acagcatgctggggagcctgcagtctgacctgaacaagctgggggtcgccacagtcgcc
aaaggagtctgcggggcctgcaagaagcccatcgccgggcaggttgtgaccgccatggg
gaagacgtggcaccccgagcacttcgtctgcacccactgccaggaggagatcggatccc
ggaacttcttcgagcgggatggacagccctactgtgaaaaggactaccacaacctcttc
tccccgcgctgctactactgcaacggccccatcctggataaagtggtgacagcccttga
ccggacgtggcaccctgaacacttcttctgtgcacagtgtggagccttctttggtcccg
aagggttccacgagaaggacggcaaggcctactgtcgcaaggactacttcgacatgttc
gcacccaagtgtggcggctgcgcccgggccatcctggagaactatatctcagccctcaa
cacgctgtggcatcctgagtgctttgtgtgccgggaatgcttcacgccattcgtgaacg
gcagcttcttcgagcacgacgggcagccctactgtgaggtgcactaccacgagcggcgc
ggctcgctgtgttctggctgccagaagcccatcaccggccgctgcatcaccgccatggc
caagaagttccaccccgagcacttcgtctgtgccttctgcctcaagcagctcaacaagg
gcaccttcaaggagcagaacgacaagccttactgtcagaactgcttcctcaagctcttc
tgctag

The full length nucleic acid molecule encodes paxillin iso form 1 (see Genbank Accession NP_{—}001074324, which is hereby incorporated by reference in its entirety), which has the amino acid sequence of SEQ ID NO: 22 as follows:

MDDLDALLAD LESTTSHISK RPVFLSEETP YSYPTGNHTY QEIAVPPPVP PPPSSEALNG
TILDPLDQWQ PSSSRFIHQQ PQSSSPVYGS SAKTSSVSNP QDSVGSPCSR VGEEEHVYSF
PNKQKSAEPS PTVMSTSLGS NLSELDRLLL ELNAVQHNPP GFPADEANSS PPLPGALSPL
YGVPETNSPL GGKAGPLTKE KPKRNGGRGL EDVRPSVESL LDELESSVPS PVPAITVNQG
EMSSPQRVTS TQQQTRISAS SATRELDELM ASLSDFKIQG LEQRADGERC WAAGWPRDGG
RSSPGGQDEG GFMAQGKTGS SSPPGGPPKP GSQLDSMLGS LQSDLNKLGV ATVAKGVCGA
CKKPIAGQVV TAMGKTWHPE HFVCTHCQEE IGSRNFFERD GQPYCEKDYH NLFSPRCYYC
NGPILDKVVT ALDRTWHPEH FFCAQCGAFF GPEGFHEKDG KAYCRKDYFD MFAPKCGGCA
RAILENYISA LNTLWHPECF VCRECFTPFV NGSFFEHDGQ PYCEVHYHER RGSLCSGCQK
PITGRCITAM AKKFHPEHFV CAFCLKQLNK GTFKEQNDKP YCQNCFLKLF C

The serine-83, serine-126, and serine-130 residues are shown in bold and underlined.

This nucleic acid molecule also encodes a splice variant of paxillin, isoform 2 (see Genbank Accession NP_{—}002850, which is hereby incorporated by reference in its entirety), which has the amino acid sequence of SEQ ID NO: 23 as follows:

MDDLDALLAD LESTTSHISK RPVFLSEETP YSYPTGNHTY QEIAVPPPVP PPPSSEALNG
TILDPLDQWQ PSSSRFIHQQ PQSSSPVYGS SAKTSSVSNP QDSVGSPCSR VGEEEHVYSF
PNKQKSAEPS PTVMSTSLGS NLSELDRLLL ELNAVQHNPP GFPADEANSS PPLPGALSPL
YGVPETNSPL GGKAGPLTKE KPKRNGGRGL EDVRPSVESL LDELESSVPS PVPAITVNQG
EMSSPQRVTS TQQQTRISAS SATRELDELM ASLSDFKFMA QGKTGSSSPP GGPPKPGSQL
DSMLGSLQSD LNKLGVATVA KGVCGACKKP IAGQVVTAMG KTWHPEHFVC THCQEEIGSR
NFFERDGQPY CEKDYHNLFS PRCYYCNGPI LDKVVTALDR TWHPEHFFCA QCGAFFGPEG
FHEKDGKAYC RKDYFDMFAP KCGGCARAIL ENYISALNTL WHPECFVCRE CFTPFVNGSF
FEHDGQPYCE VHYHERRGSL CSGCQKPITG RCITAMAKKF HPEHFVCAFC LKQLNKGTFK
EQNDKPYCQN CFLKLFC

Isoform 2 is encoded by a nucleic acid molecule lacking the bold/italicized sequence in SEQ ID NO: 21 above. The serine-83, serine-126, and serine-130 residues are shown in bold and underlined.

This nucleic acid molecule also encodes a variant of paxillin, isoform 3 (see Genbank Accession NP_{—}079433, which is hereby incorporated by reference in its entirety), which has the amino acid sequence of SEQ ID NO: 24 as follows:

MSTSLGSNLS ELDRLLLELN AVQHNPPGFP ADEANSSPPL PGALSPLYGV PETNSPLGGK
AGPLTKEKPK RNGGRGLEDV RPSVESLLDE LESSVPSPVP AITVNQGEMS SPQRVTSTQQ
QTRISASSAT RELDELMASL SDFKFMAQGK TGSSSPPGGP PKPGSQLDSM LGSLQSDLNK
LGVATVAKGV CGACKKPIAG QVVTAMGKTW HPEHFVCTHC QEEIGSRNFF ERDGQPYCEK
DYHNLFSPRC YYCNGPILDK VVTALDRTWH PEHFFCAQCG AFFGPEGFHE KDGKAYCRKD
YFDMFAPKCG GCARAILENY ISALNTLWHP ECFVCRECFT PFVNGSFFEH DGQPYCEVHY
HERRGSLCSG CQKPITGRCI TAMAKKFHPE HFVCAFCLKQ LNKGTFKEQN DKPYCQNCFL
KLFC

Isoform 3 is encoded by a nucleic acid molecule having the bold typeface ATG as the start codon in SEQ ID NO: 21 above. None of the serine residues identified above appear in iso form 3; therefore, this iso form can be used for negative selection during SELEX to remove non-specific binding aptamers.

Aptamers that are specific for non-serine phosphorylated paxillin can be delivered using substantially the same delivery systems shown in FIGS. 1-3, except that the RNAi can be replaced with the non-phosphoserine binding aptamers.

Antibodies or binding fragments thereof that can bind specifically to non-serine phosphorylated (Ser-83, Ser-126, and/or Ser-130) paxillin can also be used to inhibit the phosphorylation thereof and, thus, the activity of serine-phosphorylated form. Antibodies can be raised according to standard procedures, including both monoclonal and polyclonal antibodies.

Polyclonal antibodies can be raised by immunizing an animal (e.g., a rabbit, rat, mouse, etc.) with multiple subcutaneous or intraperitoneal injections of the relevant antigen, e.g., an isolated paxillin polypeptide fragment, paxillin fusion protein, or immunogenic conjugate) diluted in sterile saline and combined with an adjuvant (e.g., Complete or Incomplete Freund's Adjuvant) to form a stable emulsion. The isolated paxillin polypeptide fragment, paxillin fusion protein, or immunogenic conjugate includes a region of SEQ ID NO: 22 or 23 that contains serine residue 83 or one or both of serine residues 126 and 130 in an unphosphorylated form. Exemplary fragments comprise at least 20 consecutive amino acids, more preferably at least 30, 40, 50, 60, 70, or 80 consecutive amino acids. In certain embodiments, the polypeptide fragment excludes any sequence contained within isoform 3 (SEQ ID NO: 24). In certain other embodiments, the polypeptide fragment contains less than 50, 40, or 30 contiguous amino acids of SEQ ID NO: 24, more preferably less than 20 or 10 contiguous amino acids of SEQ ID NO: 24.

Regardless of the specific antigen used, following the desired immunization protocol, the polyclonal antibody is then recovered from blood or ascites of the immunized animal. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc. Polyclonal antiserum can also be rendered monospecific using standard procedures (see e.g., Agaton et al., “Selective Enrichment of Monospecific Polyclonal Antibodies for Antibody-Based Proteomics Efforts,” J Chromatography A 1043(1):33-40 (2004), which is hereby incorporated by reference in its entirety). The specificity of the polyclonal antiserum for non-phosphorylated serine residues, particularly residues 83 and 126/130, can be screened using appropriate immunoassays.

Monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety. Using the hybridoma method, a mouse, hamster, or other appropriate host animal, is immunized to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against paxillin, as determined by immunoprecipitation, immunoblotting, or by an in vitro binding assay such as radioimmunoassay (RIA) or enzyme-linked immunosorbent assay (ELISA) can then be propagated either in in vitro culture using standard methods (JAMES W. GODING, MONOCLONAL ANTIBODIES: PRINCIPLES AND PRACTICE (Academic Press 1986), which is hereby incorporated by reference in its entirety) or in vivo as ascites tumors in an animal. The monoclonal antibodies can then be purified from the culture medium or ascites fluid as described for polyclonal antibodies above. The specificity of the monoclonal antibodies for non-phosphorylated serine residues, particularly residues 83 and 126/130, can be screened using appropriate immunoassays.

Alternatively monoclonal antibodies can also be made using recombinant DNA methods as described in U.S. Pat. No. 4,816,567 to Cabilly et al, which is hereby incorporated by reference in its entirety. Polynucleotides encoding a monoclonal antibody are isolated, from mature B-cells or hybridoma cell, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries as described (McCafferty et al., “Phage Antibodies: Filamentous Phage Displaying Antibody Variable Domains,” Nature 348:552-554 (1990); Clackson et al., “Making Antibody Fragments Using Phage Display Libraries,” Nature, 352:624-628 (1991); and Marks et al., “By-passing Immunization. Human Antibodies from V-gene Libraries Displayed on Phage,” J Mol Biol 222:581-597 (1991), which are hereby incorporated by reference in their entirety).

The isolated antibodies of the present invention may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The isolated antibody can be a full length antibody, monoclonal antibody (including full length monoclonal antibody), polyclonal antibody, multispecific antibody (e.g., bispecific antibody), human, humanized or chimeric antibody, or antibody fragments, so long as they exhibit the desired activity, e.g., an ability to inhibit serine phosphorylation of paxillin so as to inhibit nuclear translocation of serine-phosphorylated paxillin.

Examples of suitable binding fragments include, without limitation, Fab fragments, F(ab)₂ fragments, Fab′ fragments, F(ab′)₂ fragments, Fd fragments, Fd′ fragments, Fv fragments, and minibodies, e.g., 61-residue subdomains of the antibody heavy-chain variable domain (Pessi et al., “A Designed Metal-binding Protein with a Novel Fold,” Nature, 362:367-369 (1993), which is hereby incorporated by reference in its entirety). Domain antibodies (dAbs) (see, e.g., Holt et al., “Domain Antibodies: Proteins for Therapy,” Trends Biotechnol. 21:484-90 (2003), which is hereby incorporated by reference in its entirety) are also suitable for the methods of the present invention. These antibody fragments can be made by conventional procedures, such as proteolytic fragmentation procedures, as described in J. Goding, MONOCLONAL ANTIBODIES: PRINCIPLES AND PRACTICE 98-118 (1984), which is hereby incorporated by reference in its entirety.

Further, single chain antibodies are also suitable for the present invention (e.g., U.S. Pat. Nos. 5,476,786 to Huston and 5,132,405 to Huston & Oppermann; Huston et al., “Protein Engineering of Antibody Binding Sites: Recovery of Specific Activity in an Anti-digoxin Single-chain Fv Analogue Produced in Escherichia coli,” Proc. Nat'l Acad. Sci. USA 85:5879-83 (1988); U.S. Pat. No. 4,946,778 to Ladner et al.; Bird et al., “Single-chain Antigen-binding Proteins,” Science 242:423-6 (1988); Ward et al., “Binding Activities of a Repertoire of Single Immunoglobulin Variable Domains Secreted from Escherichia coli,” Nature 341:544-6 (1989), each of which is hereby incorporated by reference in its entirety). Single chain antibodies are formed by linking the heavy and light immunoglobulin chain fragments of the Fv region via an amino acid bridge, resulting in a single chain polypeptide. The use of univalent antibodies are also embraced by the present invention.

As noted above, the monoclonal antibody of the present invention can be a humanized antibody. Humanized antibodies are antibodies that contain minimal sequences from non-human (e.g. murine) antibodies within the variable regions. Such antibodies are used therapeutically to reduce antigenicity and human anti-mouse antibody responses when administered to a human subject. An antibody can be humanized by substituting the complementarity determining region (CDR) of a human antibody with that of a non-human antibody (e.g. mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

These antibodies can be delivered to targeted cancer cells using modifications of the coupling procedures described for the RNAi delivery systems of FIGS. 1-3, instead using appropriate peptide coupling chemistry.

Antibody mimics that specifically bind to non-serine phosphorylated paxillin and inhibit nuclear transport of paxillin can also be utilized. A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Nat'l Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety). Variations in these antibody mimics can be created by substituting one or more domains of these polypeptides and then screening the modified monobodies or affibodies for non-serine phosphorylated paxillin binding and inhibitory activity. These antibody mimics can be delivered using similar strategies as described for aptamer and antibody delivery.

As demonstrated in the accompanying examples, paxillin plays a role in fostering both androgen-dependent and androgen-independent growth and/or proliferation of prostate cancer cells, and disruption of paxillin expression or function (e.g., nuclear transport of serine-phosphorylated paxillin) can inhibit paxillin-mediated growth and/or proliferation of both androgen-dependent and androgen-independent prostate cancer cells. Therefore, the methods of treatment contemplated here can be used during treatment of cancers while they remain steroid-dependent as well as during treatment of cancers that are steroid-independent. As used herein, “proliferation” means the growth of cell population through cell division Inhibition of growth and/or proliferation means to reduce the in vitro or in vivo rate of either steroid-dependent or steroid-independent cancer cell proliferation, preferably by at least 30%, 40%, or 50%, more preferably at least 60%, 70%, 80% or more, and preferably without reducing the survival of non-target (i.e., non-cancer) cells by more than 25%, more preferably no by more than 20%, 15%, or 10%.

The therapeutic agents described herein for the inhibition of cancer cell growth and/or proliferation may be administered systemically or locally. For example, systemic administration can be achieved via any parenteral route, including orally, topically, subcutaneously, intraperitoneally, intramuscularly, intranasally, and intravenously. Repeated administration of the therapeutic agents can be used. More than one route of administration can be used simultaneously, e.g., intravenous administration in association with intratumor injection. Examples of parenteral dosage forms include aqueous solutions of the active agent, in an isotonic saline, 5% glucose or other well-known pharmaceutically acceptable excipient.

An effective amount is that amount which will inhibit paxillin expression and/or nuclear translocation of serine-phosphorylated paxillin in targeted cancer cells. A given effective amount will vary from patient to patient, and in certain instances may vary with the extent of the cancer being treated. Accordingly, a given effective amount will be best determined at the time and place through routine experimentation and optimization. When administered systemically, an amount between 0.01 and 100 mg per kg body weight per day, but preferably about 0.1 to 10 mg per kg, will effect a desired therapeutic result in most instances.

The treatments contemplated here can be used both to kill targeted cancer cells as well as to inhibit the rate of growth or proliferation. Therefore, treatment of the cancer includes both a reduction of tumor size and volume, involving the killing of tumor cells, as well as a reduction in the rate of tumor growth, which involves slowing the rate of cancer cell growth and proliferation.

The therapeutic agents of the present invention can also be administered in combination with other cancer therapies, including chemotherapies, radiotherapies, immunotherapies, and surgical procedures as is well known in the art.

### EXAMPLES

The following examples are provided to illustrate embodiments of the present invention but are by no means intended to limit its scope.

### Materials and Methods for Examples 1-8

Cell Lines and Culture

LnCAP and PC3 cell lines were obtained from ATCC and cultured in RPMI 1640 medium (Invitrogen) containing 10% FBS and 1% penicillin-streptomycin. For experiments involving pharmacological inhibitors, cells were treated overnight with serum-free, phenol red-free RPMI 1640 media. Thereafter, cells were treated with vehicle (0.1% DMSO) or inhibitors Galardin, PP2, AG1478 (Calbiochem), flutamide, or erlotinib (Sigma) for 30 minutes before stimulation with 0.1% ethanol (vehicle) or 25 nM DHT for 30 minutes.

EGFR Transactivation Assay

A431 cells were used to detect DHT-mediated release of EGFR ligands from LnCAP cells. A431 cells (ATCC) were cultured in DMEM/F-12 (1:1) medium (Invitrogen) containing 10% FBS and 1% penicillin-streptomycin, serum-starved overnight, and then stimulated with medium from DHT−, DHT+Galardin−, or vehicle (0.1% ethanol)-treated LnCAP cells for 60 minutes. As controls, A431 cells were stimulated with DHT or media alone. Thereafter, A431 cells were isolated for Western blot analysis to detect phosphorylated and total EGFR.

Transient Transfection

PC3 or LnCAP cells were treated with non-targeting siRNA pool (ThermoFisher Scientific) or paxillin-specific siRNA according to manufacturer's instructions. Two sets of human paxillin siRNAs were used: 1) human paxillin siRNA (Santa Cruz Biotechnology) containing three target-specific 20-25 nucleotide siRNAs or 2) human paxillin siRNA ON-TARGET plus SMARTpoo1 (ThemoFisher Scientific) containing four siRNAs targeting the paxillin mRNA. The latter was used for all experiments described in the accompanying Examples, although results were similar with both pools. For experiments involving constitutively active (“CA”) Raf (William Walker, University of Pittsburgh) or MEK (Melanie Cobb, University of Texas Southwestern Medical Center), cells were co-transfected with paxillin or nonspecific siRNAs and cDNAs encoding caRaf or caMEK. After 72 hours, cells were treated overnight with serum-free, phenol red-free RPMI 1640 media and stimulated with 0.1% ethanol/DMSO (vehicle), 25 nM DHT (Steraloids), 20 ng/ml EGF (BD Biosciences), or 100 nM PMA (Sigma) for the times indicated for Western blots or 24 hours for MTT assays.

Plasmids and Cloning

RNA was isolated from HEK293 cells using RNeasy mini kit (Qiagen) according to the manufacturer's instructions and reverse-transcribed to obtain cDNA. Paxillin was amplified from the cDNA with high fidelity Pfu Turbo (Stratagene) using primer pairs: 5′-ACCTTGAATTCATG-GACGACCTCGACGCCCTGCTGGC-3′ (SEQ ID NO: 25) and 5′-CTAAGC-GGCCGCTTACTAGCAGAAGAGCTTGAGGAAGCA-3′ (SEQ ID NO: 26). Wild-type paxillin was then cloned into pcDNA3.1(+) plasmid (Invitrogen) and confirmed by sequencing.

Site-Directed Mutagenesis

Site-directed mutagenesis (Stratagene) was used to convert serine residues 83, 126, and 130 (583A/S126A/S130A) or tyrosine residues 31 and 118 (Y31A/Y188A) to alanine. Clones were sequenced in entirety to confirm mutations. Residues were chosen based on previous studies (Brown and Turner, “Paxillin: Adapting to Change,” Physiol. Rev. 84(4):1315-1339 (2004); Deakin and Turner, “Paxillin Comes of Age,” J. Cell Sci. 121:2435-2444 (2008); Woodrow et al., “Ras-Induced Serine Phosphorylation of the Focal Adhesion Protein Paxillin is Mediated by the Raf-->MEK-->ERK Pathway,” Exp. Cell Res. 287(2):325-338 (2003); Cai et al., “Glycogen Synthase Kinase 3- and Extracellular Signal-Regulated Kinase-Dependent Phosphorylation of Paxillin Regulates Cytoskeletal Rearrangement,” Mol. Cell. Biol. 26(7):2857-2868 (2006), which are hereby incorporated by reference in their entirety), demonstrating their importance for paxillin function.

Paxillin Rescue Experiments

PC3 cells were transfected with paxillin siRNA as described above. After 96 hours, media was removed, and cells were transfected with Lipofectamine (Invitrogen) or Lipofectamine plus WT, S83A/S126A/S130A, or Y31A/Y118A paxillin cDNA. After 48 hours, cells were treated overnight with serum-free, phenol red-free RPMI 1640 media and stimulated with the indicated ligands for 30 minutes for Western blots or 24 hours for MTT assays.

Western Blot Analysis

Western blots were performed as described (Evaul and Hammes, “Cross-Talk Between G Protein-Coupled and Epidermal Growth Factor Receptors Regulates Gonadotropin-Mediated Steroidogenesis in Leydig Cells,” J. Biol. Chem. 283(41):27525-27533 (2008), which is hereby incorporated by reference in its entirety). Primary antibodies were: paxillin, paxillinphospho-Tyr118, paxillin-phospho-Tyr31, Akt1/2/3, Akt1/2/3 phospho-Ser473, EGFR at 1:1000 dilution, and EGFR phospho-Tyr845 (1:500) (Santa Cruz Biotechnology); paxillin phospho-Ser126 (Invitrogen); paxillin phospho-Ser83 (ECM Biosciences); p44/42-Erk1/2, phospho-p44/42-Erk1/2 (Thr202/Tyr04), phospho-Ser217/221-MEK1/2, src, src-phospho-Tyr416 (Cell Signaling Technology) at 1:1000 dilutions and f3-actin (1:5000, Millipore).

MTT Assay

MTT assays were performed using a colorimetric assay cell proliferation kit (Roche Applied Science) according to the manufacturer's instructions.

Cell Migration/Invasion Assay

Cell migration/invasion assays were performed using a colorimetric QCM Cell invasion assay kit containing 24-well Boyden chambers with extracellular matrix-coated 8-sm pore size membranes (Millipore) according to the manufacturer's instructions.

RNA Extraction and Real Time PCR

RNA was isolated with the RNeasy mini kit (Qiagen) according to manufacturer's instructions. Levels of Kallikrein-related peptidase-3 (KLK3, or prostate-specific antigen (“PSA”)), paxillin, cyclin D1, and GAPDH expression were analyzed by the A/A-Ct method using Taqman gene expression assay primers (assay ID# Hs99999905_ml-GAPDH, Hs02576345_ml-KLK3, Hs99999004_ml-CCND1, and Hs01104424-ml-PXN; Applied Biosystems) on an ABI StepOne plus real-time PCR machine.

Statistical Analysis

Results for the MTT assay, cell migration-invasion assay, and real-time PCR were analyzed using Student's t test. A value of p≦0.05 was considered significant.

### Example 1

### DHT-Induced Erk Signaling Occurs Via Matrix Metalloproteinase (MMP)-Mediated Transactivation of the EGF Receptor

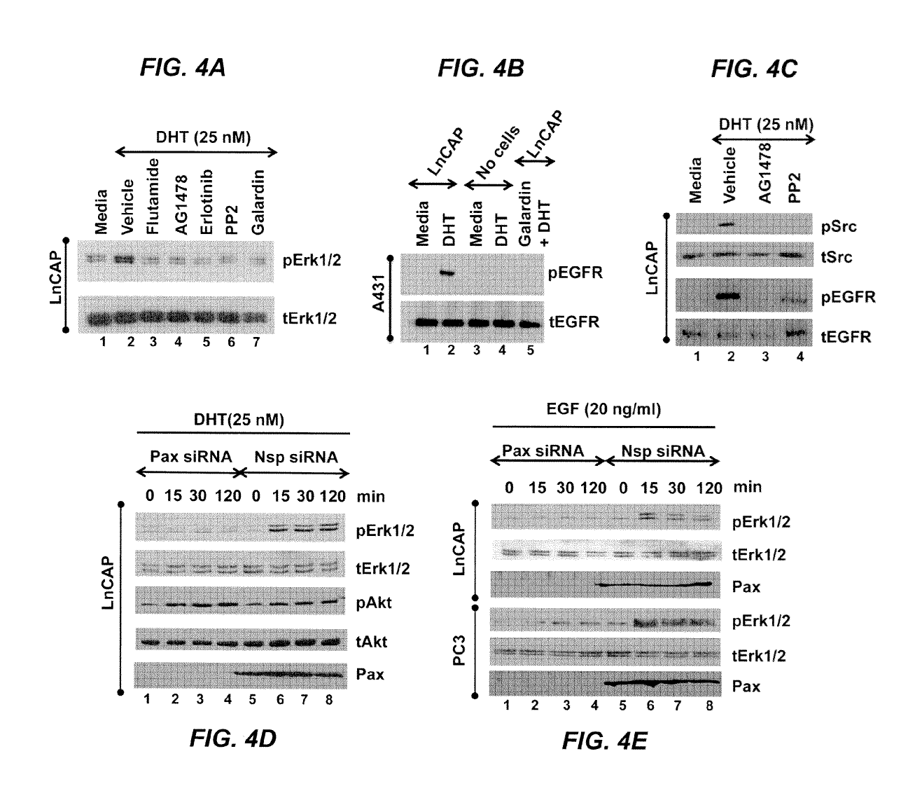

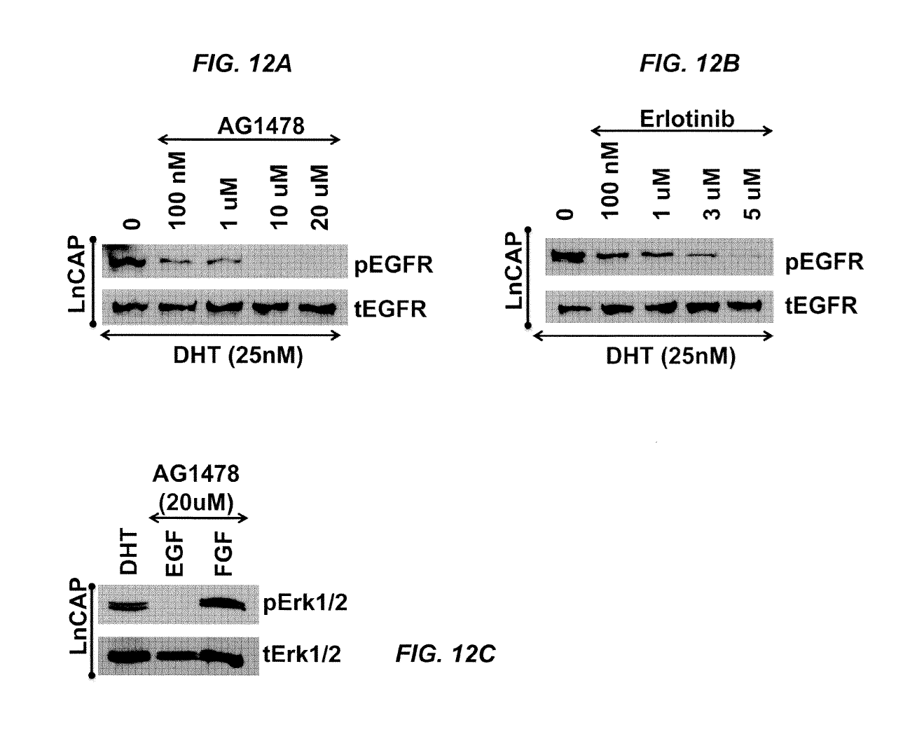

Because androgens induce Erk1/2 signaling in PCa cell lines (Migliaccio et al., “Crosstalk Between EGFR and Extranuclear Steroid Receptors,” Ann. N.Y. Acad. Sci. 1089:194-200 (2006); Peterziel et al., “Rapid Signaling by Androgen Receptor in Prostate Cancer Cells,” Oncogene 18(46):6322-6329 (1999); Migliaccio et al., “Steroid-Induced Androgen Receptor-Oestradiol Receptor Beta-Src Complex Triggers Prostate Cancer Cell Proliferation,” EMBO J. 19(20):5406-5417 (2000), which are hereby incorporated by reference in their entirety) the underlying mechanism by which DHT activates Erk1/2 in LnCAP cells was examined. DHT treatment of LnCAP cells for 30 minutes significantly induced Erk1/2 phosphorylation/activation (FIG. 4A, lane 2). Notably, saturating concentrations of DHT (25 nM) were used in these studies to max-imize the significance of the inhibitor effects; however, lower concentrations (1-10 nM) promoted a similar magnitude response. DHT levels in the prostate have not been accurately determined but are reported to be at least 10-20 nM (Titus et al., “Testosterone and Dihydrotestosterone Tissue Levels in Recurrent Prostate Cancer,” Clin. Cancer Res. 11(13):4653-4657 (2005); Nishiyama et al., “The Change in the Dihydrotestosterone Level in the Prostate Before and After Androgen Deprivation Therapy in Connection with Prostate Cancer Aggressiveness Using the Gleason Score,” J. Urol. 178:1282-1288 (2007), which are hereby incorporated by reference in their entirety). Pretreatment with the androgen receptor antagonist flutamide (FIG. 4A, lane 3) as well as EGF receptor inhibitors AG1478 and Erlotinib (FIG. 4A, lanes 4 and 5, respectively) blocked Erk1/2 phosphorylation, indicating that DHT induces EGFR transactivation and subsequent Erk1/2 signaling via classical androgen receptors. The concentrations of AG1478 (20 μM) and erlotinib (5 μM) used were based on con-centration gradient experiments (FIG. 12A-B) and previous studies in PCa cells and other cell lines. Of note, AG1478 at 20 μM specifically blocks EGF but not FGF-induced Erk activation in LnCAP (FIG. 12C) and MEK activation in MLTC (Evaul and Hammes, “Cross-Talk Between G Protein-Coupled and Epidermal Growth Factor Receptors Regulates Gonadotropin-Mediated Steroidogenesis in Leydig Cells,” J. Biol. Chem. 283(41):27525-27533 (2008), which is hereby incorporated by reference in its entirety) cells, thus, demonstrating the specificity of AG1478 to EGFR inhibition and ruling out off-target effects on the Ras/Raf/MEK/Erk pathway. Finally, the inhibitors alone (in absence of DHT) had no effect on Erk signaling.

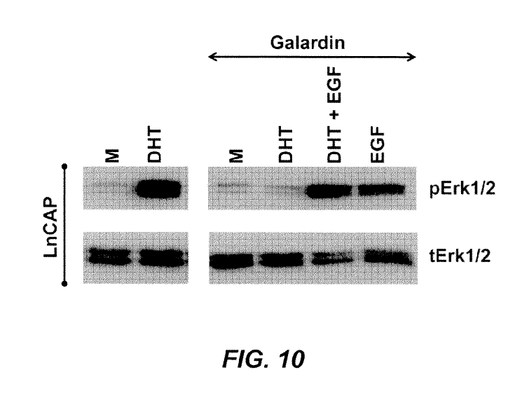

The ability of the MMP inhibitor galardin to block DHT-induced Erk1/2 phosphorylation (FIG. 4A, lane 7) and its rescue by EGF treatment (FIG. 10) demonstrates that DHT activates the EGFR through MMP activation, possibly by release of membrane-associated EGFR ligands (Razandi et al., “Proximal Events in Signaling by Plasma Membrane Estrogen Receptors,” J. Biol. Chem. 278(4):2701-2712 (2003); Filardo et al., “Estrogen Action Via the G Protein-Coupled Receptor, GPR30: Stimulation of Adenylyl Cyclase and cAMP-Mediated Attenuation of the Epidermal Growth Factor Receptor-to-MAPK Signaling Axis,” Mol. Endocrinol. 16(1):70-84 (2002), which are hereby incorporated by reference in their entirety). In fact, medium from DHT- (FIG. 4B, lane 2) but not vehicle-treated (FIG. 4B, lane 1) LnCAP cells increased EGF receptor phosphorylation in A431 cells, which are known to express very high levels of EGFR, indicating that EGF receptor ligands were being released into the medium from DHT-treated LnCAP cells. Moreover, the addition of galardin to DHT-treated LnCAP cells (FIG. 4B, lane 5) blocked the ability of the medium to activate EGF receptor signaling in the A431 cells, demonstrating that the release of EGF receptor ligands is indeed MMP-dependent. Finally, DHT alone (FIG. 4B, lane 4) did not promote EGF receptor activation in the A431 cells.

Once activated, phosphorylated EGFR (FIG. 4C, lane 2) stimulates Src, as the EGFR inhibitor AG 1478 blocked both Src (FIG. 4C, lane 3) and Erk (FIG. 4A, lane 4) phosphorylation, whereas the Src inhibitor PP2 blocked Erk (FIG. 4A, lane 6) but not EGFR (FIG. 4C, lane 4) phosphorylation. Thus, outside the nucleus, DHT functions just like EGF in LnCAP cells, only through indirect rather than direct activation of the EGF receptor.

### Example 2

### Paxillin Regulates DHT- and EGF-Induced ERK Activation in Prostate Cancer Cells

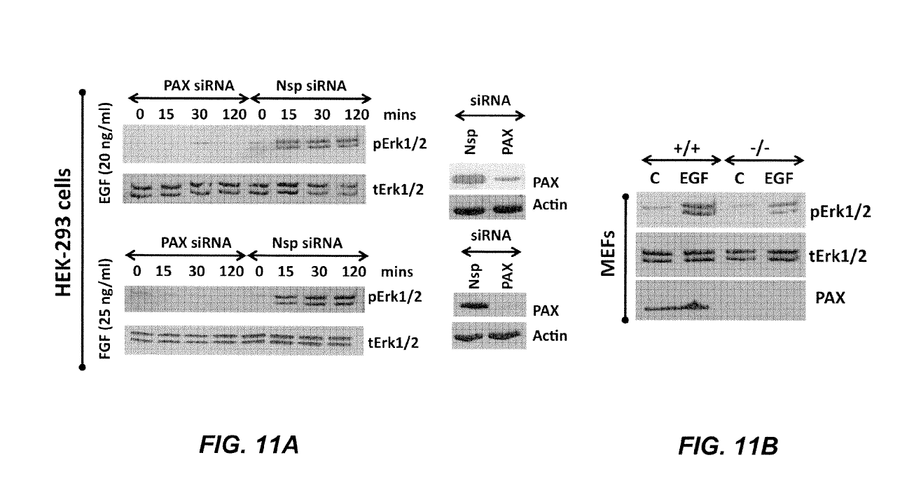

Next it was investigated whether paxillin regulated Erk1/2 activation in prostate cancer cells. In androgen-dependent LnCAP cells, both DHT (FIG. 4D) and EGF (FIG. 4E) induced Erk1/2 activation from 15 to 120 minutes. Knockdown of paxillin abrogated DHT-induced Erk1/2 activation, but not Akt activation (FIG. 4D). Furthermore, paxillin knockdown in either LnCAP or androgen-independent PC3 cells inhibited EGF-induced Erk1/2 activation (FIG. 4E) but not Akt activation. Similar effects were observed in DHT-treated LAPC-4 prostate cancer cells and FGF- or EGF-treated HEK-293 cells (FIG. 11A). These observations indicate that paxillin is an important regulator of growth factor receptor-induced Erk1/2 signaling regardless of cell type or how the growth factor receptor is activated (EGFR either indirectly by DHT or directly by EGF, or FGFR directly by FGF). Furthermore, EGF-mediated Erk activation was attenuated in mouse embryonic fibroblasts from paxillin null mice (from Dr. Sheila Thomas, Harvard University), providing genetic confirmation of the siRNA experiments that paxillin is important for Erk1/2 signaling (FIG. 11B).

### Example 3

### Paxillin Acts Downstream of the EGF Receptor but Upstream of Raf/MEK

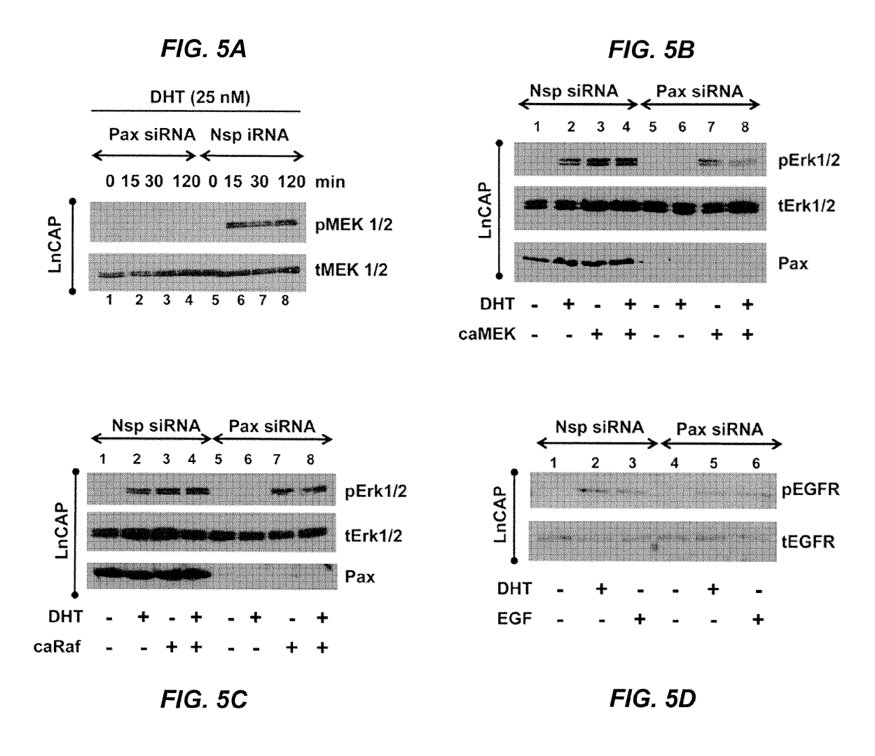

To investigate where paxillin functions in EGF receptor-induced signaling, DHT- or EGF-induced activation of MEK and EGFR in paxillin-ablated LnCAP cells was examined. Similar to Erk1/2 activation, knockdown of paxillin markedly lowered DHT- (FIG. 5A) and EGF-induced MEK1/2 phosphorylation. Furthermore, loss of DHT-induced Erk1/2 activation by paxillin knockdown could be rescued by overexpression of constitutively activated MEK (caMEK; FIG. 5B, lanes 7 and 8) or Raf (caRaf; FIG. 5C, lanes 7 and 8). The caMEK used has mutations substituting glutamic and aspartic acid for Ser-218 and Ser-222, significantly increasing the basal activity of MEK over the unphosphorylated wild-type enzyme (Robinson et al., “Contributions of the Mitogen-Activated Protein (MAP) Kinase Backbone and Phosphorylation Loop to MEK Specificity,” J. Biol. Chem. 271:29734-29739 (1996); Mansour et al., “Transformation of Mammalian Cells by Constitutively Active MAP Kinase Kinase,” Science 265(5174):966-970 (1994), which are hereby incorporated by reference in their entirety). The caRaf used is a fusion protein of the membrane localization signal of Ras to the carboxyl terminus of Raf that is constitutively activated independent of cellular Ras (Leevers et al., “Comparative Effects of DHEA vs. Testosterone, Dihydrotestosterone, and Estradiol on Proliferation and Gene Expression in Human LNCaP Prostate Cancer Cells,” Am. J. Physiol. Endocrinol. Metab. 288(3):E573-E584 (2005), which is hereby incorporated by reference in its entirety). Finally, knock-down of paxillin minimally affected DHT- or EGF-induced EGFR phosphorylation (FIG. 5D). Together these results demonstrate that paxillin functions downstream of the EGFR but upstream of Raf and MEK to regulate Erk1/2 activation.

### Example 4

### Paxillin Knockdown Prevents Proliferation, Migration, and Invasion of Prostate Cancer Cells

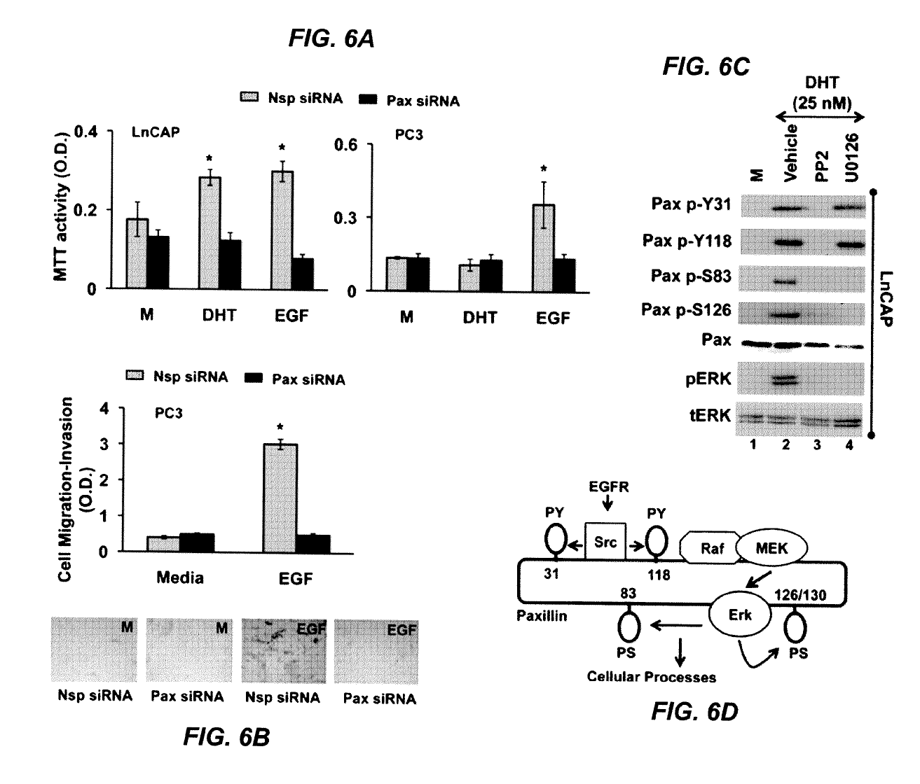

Prostate cancer progression correlates with proliferation, migration, and invasion, all of which are thought to require Erk1/2 signaling (Hammes and Levin, “Extranuclear Steroid Receptors: Nature and Actions,” Endocr. Rev. 28(7):726-741 (2007); Unni et al., “Changes in Androgen Receptor Nongenotropic Signaling Correlate with Transition of LNCaP Cells to Androgen Independence,” Cancer Res. 64(19):7156-7168 (2004); Migliaccio et al., “Steroid-Induced Androgen Receptor-Oestradiol Receptor Beta-Src Complex Triggers Prostate Cancer Cell Proliferation,” EMBO J. 19(20):5406-5417 (2000), which are hereby incorporated by reference in their entirety). Thus, to establish the physiologic importance of paxillin in prostate cancer cells, LnCAP or PC3 cells were treated with nonspecific or paxillin-specific siRNAs and measured DHT- or EGF-induced cell proliferation by MTT assay. DHT (25 nM) and EGF (20 ng/ml) significantly stimulated cell proliferation in LnCAP cells (FIG. 6A). Consistent with previous studies (Leevers et al., “Comparative Effects of DHEA vs. Testosterone, Dihydrotestosterone, and Estradiol on Proliferation and Gene Expression in Human LNCaP Prostate Cancer Cells,” Am. J. Physiol. Endocrinol. Metab. 288(3):E573-E584 (2005); Zheng et al., “SUMO-3 Enhances Androgen Receptor Transcriptional Activity through a Sumoylation-Independent Mechanism in Prostate Cancer Cells,” J. Biol. Chem. 281(7):4002-4012 (2006); Fu et al., “Hormonal Control of Androgen Receptor Function through SIRT1,” Mol. Cell. Biol. 26(21):8122-8135 (2006); Gamble et al., “Androgens Target Prohibitin to Regulate Proliferation of Prostate Cancer Cells,” Oncogene 23(17):2996-3004 (2004); Ripple et al., “Prooxidant-Antioxidant Shift Induced by Androgen Treatment of Human Prostate Carcinoma Cells,” J. Natl. Cancer Inst. 89(1):40-48 (1997), which are hereby incorporated by reference in their entirety), it was found that 0.1-100 nM DHT promoted LnCAP proliferation by both MTT and BrdU incorporation under conditions used here. Knockdown of paxillin markedly reduced DHT- and EGF-stimulated cell proliferation (FIG. 6A, left panel). Because PC3 cells are androgen-independent, EGF, but not DHT, induced cell proliferation (FIG. 6A, right panel) that was also abrogated by paxillin knockdown.

Next, an in vitro cell migration/invasion assay was used, consisting of a 24-well Boyden chamber with an extracellular matrix-coated membrane to investigate the importance of paxillin for EGF-induced cell migration and invasion of PC3 cells. PC3 cells migrated through the membrane and invaded the matrix in response to EGF, and paxillin knockdown abrogated this physiological process. FIG. 6B demonstrates quantitative analysis of migration/invasion by measuring absorbance after staining invading cells (upper panel) as well as qualitative images of the extracellular matrix-coated membrane underside containing migrated and invaded cells (lower panel). Collectively, these data demonstrate that paxillin is critical for proliferation, migration, and invasion of prostate cancer cell lines.

### Example 5

### Phosphorylation of Paxillin at Tyrosines 31/118 and Serines 83/126/130 is Essential for DHT- and EGF-Mediated Prostate Cancer Cell Proliferation

Receptor-tyrosine kinases are known to promote tyrosine and serine phosphorylation of paxillin (Brown and Turner, “Paxillin: Adapting to Change,” Physiol. Rev. 84(4):1315-1339 (2004); Woodrow et al., “Ras-Induced Serine Phosphorylation of the Focal Adhesion Protein Paxillin is Mediated by the Raf->MEK->ERK Pathway,” Exp. Cell Res. 287(2):325-338 (2003); Cai et al., “Glycogen Synthase Kinase 3- and Extracellular Signal-Regulated Kinase-Dependent Phosphorylation of Paxillin Regulates Cytoskeletal Rearrangement,” Mol. Cell. Biol. 26(7):2857-2868 (2006); Ishibe et al., “Paxillin Serves as an ERK-Regulated Scaffold for Coordinating FAK and Rac Activation in Epithelial Morphogenesis,” Mol. Cell. 16(2):257-267 (2004); Ishibe et al., “Phosphorylation-Dependent Paxillin-ERK Association Mediates Hepatocyte Growth Factor-Stimulated Epithelial Morphogenesis,” Mol. Cell. 12(5):1275-1285 (2003); Chen et al., “Brk Activates Racl and Promotes Cell Migration and Invasion by Phosphorylating Paxillin,” Mol. Cell. Biol. 24(24):10558-10572 (2004); Webb et al., “Paxillin Phosphorylation Sites Mapped by Mass Spectrometry,” J. Cell Sci. 118:4925-4929 (2005), which are hereby incorporated by reference in their entirety). Results confirmed that, in LnCAP cells, DHT triggered phosphorylation of paxillin at tyrosines 31/118 and serines 83/126 (FIG. 6C, lane 2). Inhibition of the EGFR with AG1478 or Src with PP2 (FIG. 6C, lane 3) abrogated phosphorylation at all four sites (FIG. 6C, lane 3). In contrast, the MEK inhibitor U0126 blocked DHT-induced phosphorylation of paxillin at serines 83/126 but not tyrosines 31/118 (FIG. 6C, lane 4). These results demonstrate that DHT, via trans-activation of the EGFR, promotes Src-mediated phosphorylation of paxillin at tyrosines 31/118, which is then required for subsequent Erk-mediated phosphorylation of serines 83/126 (see FIG. 6D).

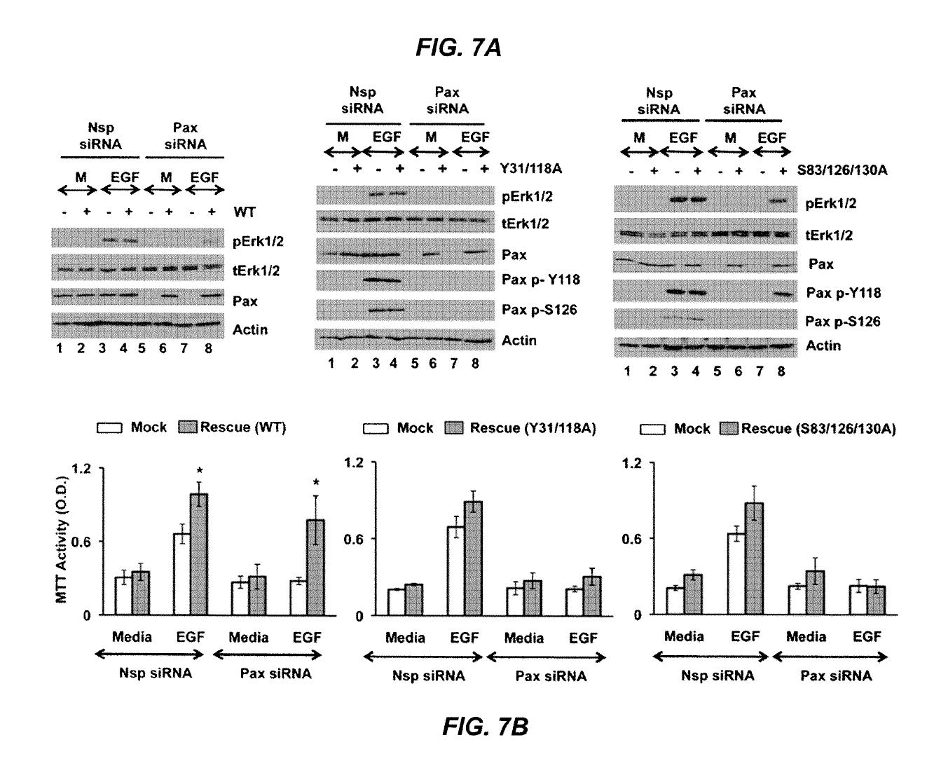

To confirm this proposed signaling sequence as well as the physiologic importance of these phosphorylation events, mutation studies were carried out to identify critical phosphorylation sites. Tyrosines 31 and 118 (Y31A/Y118A-paxillin) and serines 83,126, and 130 (S83A/S126A/S130A-paxillin) were mutated to alanines. Serine 130 was included because it is phosphorylated in conjunction with serine 126 (Unni et al., “Changes in Androgen Receptor Nongenotropic Signaling Correlate with Transition of LNCaP Cells to Androgen Independence,” Cancer Res. 64(19):7156-7168 (2004); Woodrow et al., “Ras-Induced Serine Phosphorylation of the Focal Adhesion Protein Paxillin is Mediated by the Raf->MEK->ERK Pathway,” Exp. Cell Res. 287(2):325-338 (2003); Cai et al., “Glycogen Synthase Kinase 3- and Extracellular Signal-Regulated Kinase-Dependent Phosphorylation of Paxillin Regulates Cytoskeletal Rearrangement,” Mol. Cell. Biol. 26(7):2857-2868 (2006), which are hereby incorporated by reference in their entirety). Thereafter, endogenous paxillin expression in PC3 cells was knocked down by siRNA and re-expressed WT paxillin, S83A/S126A/S130A-paxillin, or Y31A/Y118A-paxillin. As expected, re-expression of WT paxillin rescued EGF-induced Erk1/2 phosphorylation (FIG. 7A, left panel, lane 8) and cell proliferation (FIG. 7B, left panel, right-most bar). In contrast, Y31A/Y118A-paxillin failed to rescue both EGF-induced Erk1/2 activation and cell proliferation (FIG. 7A, middle panel, lane 8; and FIG. 7B, middle panel, last bar). These results confirm that Src-regulated paxillin phosphorylation of tyrosines is required for EGFR-mediated activation of Erk1/2 and downstream cellular functions. Notably, although endogenous paxillin was phosphorylated at Ser-126 in response to EGF (FIG. 7A, middle panel, lanes 3 and 4), re-expressed Y31A/Y118A-paxillin in the absence of endogenous wild-type paxillin was not (FIG. 7A, middle panel, lane 8), confirming that EGFR-mediated serine phosphorylation of paxillin requires prior tyrosine phosphorylation. Consistent with tyrosine phosphorylation being upstream of serine phosphorylation, S83A/S126A/S130A-paxillin was still phosphorylated at Tyr-118 in response to EGF (FIG. 7A, right panel, lane 8). Surprisingly, however, in the absence of endogenous paxillin, expression of S83A/S126A/S130A-paxillin rescued EGF-induced Erk1/2 activation (FIG. 7A, right panel, lane 8) but not cell proliferation (FIG. 7B, right panel, last bar). These results indicate that, although phosphorylation of paxillin at tyrosines 31/118 is essential for EGFR-mediated Erk1/2 activation and downstream cell proliferation, phosphorylation of paxillin at serines 83/126/130 is only necessary for cell proliferation.

### Example 6

### Paxillin Specifically Regulates Receptor-Tyrosine Kinase—but not PKCinduced Erk Activation

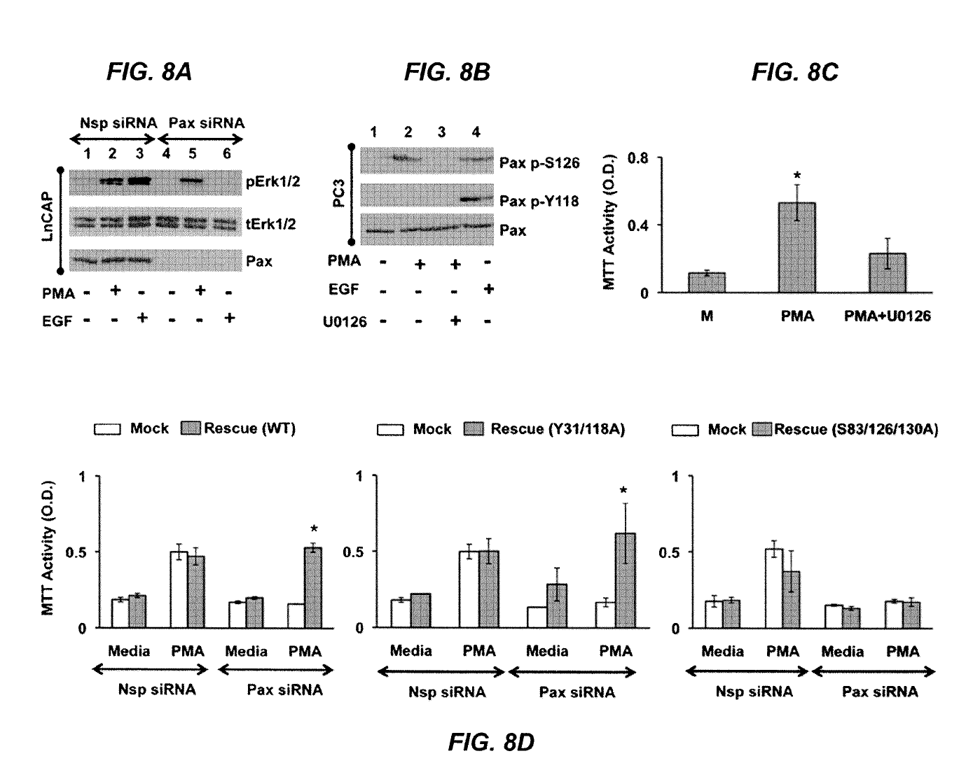

Because data indicated that paxillin was a critical regulator EGFR/Src-in-duced Erk1/2 activation, it was next determined whether paxillin was a universal modulator of Erk. As an alternative, receptor-tyrosine kinase/Src-independent means of activating Erk1/2, PMA was used to promote PKC-mediated Erk1/2 signaling. Paxillin-siRNA-treated LnCAP cells were stimulated with 0.1% DMSO (vehicle), EGF, or PMA for 30 min and Erk1/2 phosphorylation measured. Knockdown of paxillin abrogated EGF- (FIG. 8A, lane 6) but not PMA- (FIG. 8A, lane 5) induced Erk1/2 activation, demonstrating that the regulation of Erk activation by paxillin may be relatively specific to the receptor-tyrosine kinase/Src signaling pathway.

### Example 7

### PMA-Mediated Prostate Cancer Cell Proliferation Requires Erk-Mediated Phosphorylation of Paxillin

In contrast to EGF treatment of PC3 cells, which promoted Src-mediated tyrosine as well as Erk-mediated serine phosphorylation of paxillin (FIG. 8B, lane 4), PMA only promoted serine phosphorylation (FIG. 8B, lane 2). This PMA-induced serine phosphorylation of paxillin was blocked by the MEK inhibitor U0126 (FIG. 8B, lane 3), demonstrating it to be MEK/Erk dependent. Interestingly, PMA-induced proliferation of PC3 cells was also blocked by U0126 (FIG. 8C). Furthermore, siRNA-mediated ablation of paxillin abrogated PMA-induced cell proliferation of PC3 cells, which could be rescued by expression of either wild-type paxillin (FIG. 8D, left panel) or Y31A/Y118A-paxillin (FIG. 8D, middle panel) but not by S83A/S126A/S130A-paxillin (FIG. 8D, right panel). These results indicate that, although paxillin is not always required for Erk1/2 activation, Erk1/2-mediated phosphorylation of paxillin at serine residues seems critical for proliferation regardless of the agonist.

### Example 8

### Erk-Mediated Phosphorylation of Paxillin is Required for Normal Transcription in LnCAP and PC3 Cells

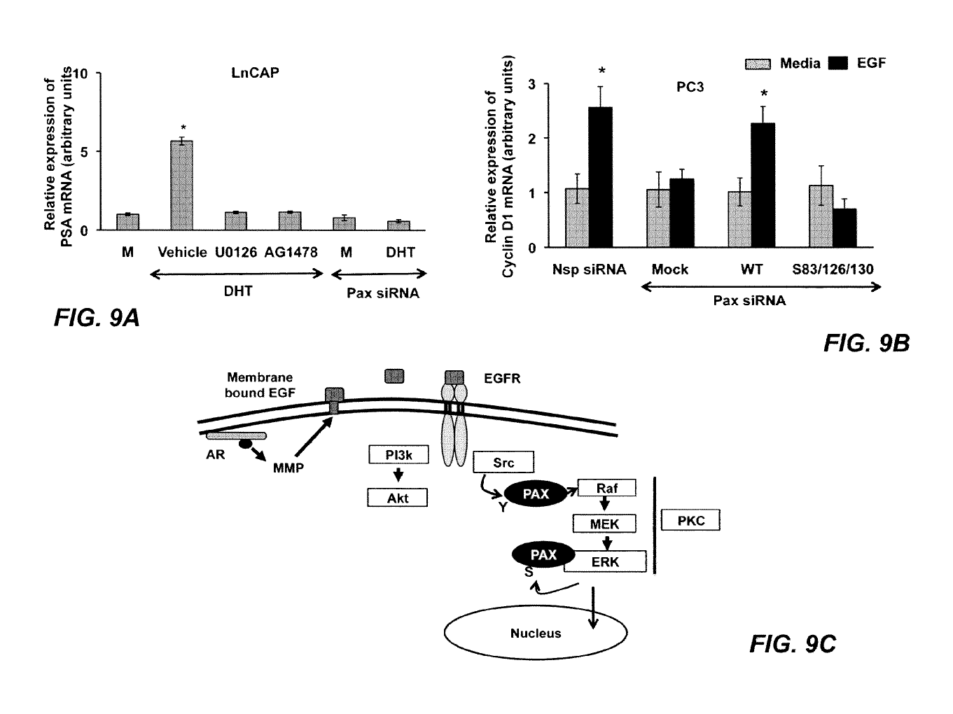

Some studies suggest that extra-nuclear kinases activated by steroids or growth factors may regulate transcription (Peterziel et al., “Rapid Signaling by Androgen Receptor in Prostate Cancer Cells,” Oncogene 18(46):6322-6329 (1999); Unni et al., “Changes in Androgen Receptor Nongenotropic Signaling Correlate with Transition of LNCaP Cells to Androgen Independence,” Cancer Res. 64(19):7156-7168 (2004); Cheng et al., “Testosterone Activates Mitogen-Activated Protein Kinase Via Src Kinase and the Epidermal Growth Factor Receptor in Sertoli Cells,” Endocrinology 148(5):2066-2074 (2007); Fix et al., “Testosterone Activates Mitogen-Activated Protein Kinase and the cAMP Response Element Binding Protein Transcription Factor in Sertoli Cells,” Proc. Natl. Acad. Sci. U.S.A. 101(30):10919-10924 (2004); Carey et al., “Ras-MEK-ERK Signaling Cascade Regulates Androgen Receptor Element-Inducible Gene Transcription and DNA Synthesis in Prostate Cancer Cells,” Int. J. Cancer 121:520-527 (2007); Franco et al., “Mitogen-Activated Protein Kinase Pathway is Involved in Androgen-Independent PSA Gene Expression in LNCaP Cells,” Prostate 56(4):319-325 (2003); Xu et al., “Androgens Induce Prostate Cancer Cell Proliferation through Mammalian Target of Rapamycin Activation and Post-Transcriptional Increases in Cyclin D Proteins,” Cancer Res. 66(15):7783-7792 (2006); O'Malley and Kumar, “Nuclear Receptor Coregulators in Cancer Biology,” Cancer Res. 69(21):8217-8222 (2009); Lidke et al., “ERK Nuclear Translocation is Dimerization-Independent but Controlled by the Rate of Phosphorylation,” J. Biol. Chem. 285(5):3092-3102 (2010), which are hereby incorporated by reference in their entirety). To examine the role of Erk and paxillin in regulating transcription in prostate cancer cells, DHT-induced expression of PSA mRNA in LnCAP cells was first studied Inhibition of Erk signaling by the MEK inhibitor U0126 or the EGFR inhibitor AG1478 as well as knockdown of paxillin expression abrogated DHT-induced expression of PSA mRNA (FIG. 9A). These data suggest that, in LnCAP cells, extra-nuclear DHT-mediated Erk1/2 activity (via EGFR and paxillin) is essential for normal intra-nuclear DHT-mediated transcription. Surprisingly, reduction of paxillin expression in PC3 cells similarly reduced EGF-mediated expression of cyclin D1 mRNA (FIG. 9B). Cyclin D1 mRNA expression could be rescued by re-expression of wild-type paxillin but not S83/126/20A-paxillin. These results, which mirror the proliferation data in FIGS. 7 and 8, indicate that paxillin may regulate Erk-induced proliferation in part by enhancing Erk1/2-mediated transcription. Thus, paxillin may help mediate cross-talk between cytoplasmic kinase and nuclear transcriptional signaling.

### Discussion of Examples 1-8

The preceding Examples reveal several novel regulatory roles of paxillin in Erk signaling that ultimately control important physiological functions in prostate cancer cells such as transcription and proliferation.

To summarize the data of the present invention, the following model is proposed to describe extra-nuclear androgen receptor-mediated signaling in prostate cancer cells (FIG. 9C). Androgens bind to classical androgen receptors, most likely located at or near the cell surface (Migliaccio et al., “Crosstalk Between EGFR and Extranuclear Steroid Receptors,” Ann. N.Y. Acad. Sci. 1089:194-200 (2006); Migliaccio et al., “Steroid-Induced Androgen Receptor-Oestradiol Receptor Beta-Src Complex Triggers Prostate Cancer Cell Proliferation,” EMBO J. 19(20):5406-5417 (2000); Cinar et al., “Phosphoinositide 3-Kinase-Independent Non-Genomic Signals Transit from the Androgen Receptor to Akt1 in Membrane Raft Microdomains,” J. Biol. Chem. 282(40):29584-29593 (2007), all of which are hereby incorporated by reference in their entirety), to promote activation of MMPs and release of membrane-associated EGF receptor ligands. Although specific EGFR ligands being released in LnCAP cells have not been identified in the present invention, previous studies implicate heparin bound-EGFs as mediators of G protein-coupled or steroid receptor cross-talk with EGF receptors (Razandi et al., “Proximal Events in Signaling by Plasma Membrane Estrogen Receptors,” J. Biol. Chem. 278(4):2701-2712 (2003); Filardo et al., “Estrogen Action Via the G Protein-Coupled Receptor, GPR30: Stimulation of Adenylyl Cyclase and cAMP-Mediated Attenuation of the Epidermal Growth Factor Receptor-to-MAPK Signaling Axis,” Mol. Endocrinol. 16(1):70-84 (2002), both of which are hereby incorporated by reference in their entirety). These ligands bind to and activate the EGFR, which then activates Src, Akt, and MEK/Erk1/2 (FIG. 9C; see also FIG. 4). Notably, prior co-immunoprecipitation studies in LnCAP cells suggested that DHT-induced Erk1/2 activation (Peterziel et al., “Rapid Signaling by Androgen Receptor in Prostate Cancer Cells,” Oncogene 18(46):6322-6329 (1999); Unni et al., “Changes in Androgen Receptor Nongenotropic Signaling Correlate with Transition of LNCaP Cells to Androgen Independence,” Cancer Res. 64(19):7156-7168 (2004), which are hereby incorporated by reference in their entirety) might be mediated by extra-nuclear steroid receptors directly binding to and activating Src (Varricchio et al., “Inhibition of Estradiol Receptor/Src Association and Cell Growth by an Estradiol Receptor Alpha Tyrosine-Phosphorylated Peptide,” Mol. Cancer. Res. 5(11):1213-1221 (2007), which is hereby incorporated by reference in its entirety) followed by EGF receptor phosphorylation (Migliaccio et al., “Crosstalk Between EGFR and Extranuclear Steroid Receptors,” Ann. N.Y. Acad. Sci. 1089:194-200 (2006); Migliaccio et al., “Steroid-Induced Androgen Receptor-Oestradiol Receptor Beta-Src Complex Triggers Prostate Cancer Cell Proliferation,” EMBO J. 19(20):5406-5417 (2000), which are hereby incorporated by reference in their entirety). However, here EGF receptor inhibition blocked DHT-induced Src phosphorylation, whereas Src inhibition had minimal effect on DHT-induced EGFR phosphorylation (FIG. 4B). Thus, similar to EGFR signaling in other cell types (Mao et al., “Activation of C-Src by Receptor Tyrosine Kinases in Human Colon Cancer Cells with High Metastatic Potential,” Oncogene 15(25):3083-3090 (1997); Goi et al., “An EGF Receptor/Ral-GTPase Signaling Cascade Regulates C-Src Activity and Substrate Specificity,” EMBO J. 19(4):623-630 (2000); Dimri et al., “Modeling Breast Cancer-Associated C-Src and EGFR Overexpression in Human MECs: C-Src and EGFR Cooperatively Promote Aberrant Three-Dimensional Acinar Structure and Invasive Behavior,” Cancer Res. 67(9):4164-4172 (2007); Frame, “Newest Findings on the Oldest Oncogene; How Activated Src Does It,” J. Cell Sci. 117:989-998 (2004); Lu et al., “The Spatiotemporal Pattern of Src Activation at Lipid Rafts Revealed by Diffusion-Corrected FRET Imaging,” PLoS Comput. Biol. 4(7):e1000127 (2008); Seong et al., “Visualization of Src Activity at Different Compartments of the Plasma Membrane by FRET Imaging,” Chem. Biol. 16(1):48-57 (2009), which are hereby incorporated by reference in their entirety), Src actions appear downstream of EGFR activation in DHT-stimulated LnCAP cells.

Irrespective of the underlying mechanism, the rapid and robust trans-activation of the EGF receptor by DHT highlights the novel concept that, outside of the nucleus, androgen actions are just like EGF with respect to activation of cytoplasmic ki-nase cascades (Migliaccio et al., “Crosstalk Between EGFR and Extranuclear Steroid Receptors,” Ann. N.Y. Acad. Sci. 1089:194-200 (2006); Hammes and Levin, “Extranuclear Steroid Receptors: Nature and Actions,” Endocr. Rev. 28(7):726-741 (2007); Cheng et al., “Testosterone Activates Mitogen-Activated Protein Kinase Via Src Kinase and the Epidermal Growth Factor Receptor in Sertoli Cells,” Endocrinology 148(5):2066-2074 (2007), which are hereby incorporated by reference in their entirety). EGFR-induced kinase pathways are known to modulate steroid receptor-mediated transcriptional signaling by altering both receptor and co-regulator activities (Peterziel et al., “Rapid Signaling by Androgen Receptor in Prostate Cancer Cells,” Oncogene 18(46):6322-6329 (1999); Unni et al., “Changes in Androgen Receptor Nongenotropic Signaling Correlate with Transition of LNCaP Cells to Androgen Independence,” Cancer Res. 64(19):7156-7168 (2004); Cheng et al., “Testosterone Activates Mitogen-Activated Protein Kinase Via Src Kinase and the Epidermal Growth Factor Receptor in Sertoli Cells,” Endocrinology 148(5):2066-2074 (2007); Fix et al., “Testosterone Activates Mitogen-Activated Protein Kinase and the cAMP Response Element Binding Protein Transcription Factor in Sertoli Cells,” Proc. Natl. Acad. Sci. U.S.A. 101(30):10919-10924 (2004); O'Malley and Kumar, “Nuclear Receptor Coregulators in Cancer Biology,” Cancer Res. 69(21):8217-8222 (2009); Lidke et al., “ERK Nuclear Translocation is Dimerization-Independent but Controlled by the Rate of Phosphorylation,” J. Biol. Chem. 285(5):3092-3102 (2010); Walker and Cheng, “FSH and Testosterone Signaling in Sertoli Cells,” Reproduction 130(1):15-28 (2005); Xu et al., “Normal and Cancer-Related Functions of the p160 Steroid Receptor Co-Activator (SRC) Family,” Nat. Rev. Cancer 9(9):615-630 (2009), which are hereby incorporated by reference in their entirety). Thus, indirect activation of the EGF receptor by DHT similarly leads to “outside-inside” cross-talk whereby rapid activation of extra-nuclear kinases enhances intra-nuclear transcriptional signaling. Data that MEK and EGFR inhibition block DHT-induced PSA mRNA expression (FIG. 9A) support this model.

Because paxillin knockdown abrogated both EGF- and DHT-induced Erk1/2 activation (FIGS. 4C-D) as well as DHT-induced PSA mRNA (FIG. 6A) and EGF-induced cyclin D1 mRNA expression (FIG. 9B), paxillin appears to be one key regulator of outside-inside signaling in response to both direct (EGF) and indirect (DHT) activation of the EGF receptor. The data further demonstrate that paxillin-mediated regulation of extra-nuclear kinases and intra-nuclear transcription in turn controls prostate cancer cell proliferation, invasion, and migration. Thus, paxillin is a critical regulator of multiple EGFR/Erk-regulated processes in prostate cancer cells.

One mechanism that explains paxillin mediation of EGFR/Erk-regulated cell functions is at the level of EGFR/Src-induced activation of the Raf/MEK/Erk signaling pathway (FIG. 9C). Prior studies suggested that Src-mediated phosphorylation of tyrosines 31/118 on paxillin was important for Erk activation in response to FAK or integrin-mediated signaling (Brown and Turner, “Paxillin: Adapting to Change,” Physiol. Rev. 84(4):1315-1339 (2004); Ishibe et al., “Paxillin Serves as an ERK-Regulated Scaffold for Coordinating FAK and Rac Activation in Epithelial Morphogenesis,” Mol. Cell. 16(2):257-267 (2004); Ishibe et al., “Phosphorylation-Dependent Paxillin-ERK Association Mediates Hepatocyte Growth Factor-Stimulated Epithelial Morphogenesis,” Mol. Cell. 12(5):1275-1285 (2003), which are hereby incorporated by reference in their entirety). The paxillin knockdown and rescue experi-ments with Y31A/Y118A-paxillin in PC3 cells unequivocally confirm that tyrosine phosphorylation of paxillin at these residues is required for EGFR-mediated Erk1/2 activation and downstream proliferation (FIG. 7). While several previous studies postulate that paxillin might function as a scaffold to hold Raf, MEK, and Erk1/2 in a signaling complex, most of these studies (Cai et al., “Glycogen Synthase Kinase 3- and Extracellular Signal-Regulated Kinase-Dependent Phosphorylation of Paxillin Regulates Cytoskeletal Rearrangement,” Mol. Cell. Biol. 26(7):2857-2868 (2006); Ishibe et al., “Paxillin Serves as an ERK-Regulated Scaffold for Coordinating FAK and Rac Activation in Epithelial Morphogenesis,” Mol. Cell. 16(2):257-267 (2004); Ishibe et al., “Phosphorylation-Dependent Paxillin-ERK Association Mediates Hepatocyte Growth Factor-Stimulated Epithelial Morphogenesis,” Mol. Cell. 12(5):1275-1285 (2003); Chen et al., “Brk Activates Racl and Promotes Cell Migration and Invasion by Phosphorylating Paxillin,” Mol. Cell. Biol. 24(24):10558-10572 (2004); Dobkin-Bekman et al., “A Preformed Signaling Complex Mediates GnRH-Activated ERK Phosphorylation of Paxillin and FAK at Focal Adhesions in L Beta T2 Gonadotrope Cells,” Mol. Endocrinol. 23(11):1850-1864 (2009); Ku and Meier, “Phosphorylation of Paxillin Via the ERK Mitogen-Activated Protein Kinase Cascade in EL4 Thymoma Cells,” J. Biol. Chem. 275(15):11333-11340 (2000), which are hereby incorporated by reference in their entirety) focused on the association of paxillin with focal adhesion molecules using overexpression and co-precipitation studies to show interactions. In contrast, in the preceding examples the morphology of prostate cancer cells with reduced paxillin expression appeared grossly normal, although cell-cell adhesions were not specifically examined. However, in paxillin knockdown cells, expression of caRaf or caMEK was sufficient to promote Erk1/2 signaling (FIG. 5B-C). Thus, although paxillin may form a complex with Raf, MEK and Erk, these interactions are not necessary for signaling by or downstream of Raf. In fact, paxillin appears to function between EGFR and Raf (FIG. 9C), as EGFR phosphorylation is minimally affected by paxillin knockdown (FIG. 5D).

Interestingly, in androgen-induced maturation of Xenopus oocytes, paxillin also functions just upstream of MOS, the germ cell homologue of Raf, again demonstrating the remarkable conservation of paxillin function from lower to higher vertebrates. However, the requirement for initial Src-mediated tyrosine phosphorylation of paxillin, the ability of paxillin to regulate downstream Erk functions regardless of the agonist, and the ability of paxillin to mediate cross-talk between cytoplasmic kinase and nuclear transcriptional signaling are all specific to somatic cells, as they are not seen in frog oocytes.

The preceding examples demonstrate that paxillin is a relatively specific regulator of receptor-tyrosine kinase/Src-mediated Erk1/2 activation in prostate cancer cells, as paxillin knockdown had no effect on PKC. Paxillin appears to be a general regulator of Erk-mediated cellular processes such as transcription or proliferation, irre-spective of the stimulus, as it was required for both PKC- and EGFR-mediated proliferation in PC3 cells (FIG. 8). Importantly, serine phosphorylation of paxillin appears critical for these Erk-mediated processes, because in paxillin-knockdown PC3 cells S83A/S126A/S130A-paxillin was unable to rescue proliferation or induction of cyclin D1 mRNA in response to either EGF- or PKC-mediated Erk1/2 activation (FIGS. 8 and 9). In fact, results of the present invention (FIGS. 6C and 8B) and previous evidence (Rasar et al., “Paxillin Regulates Steroid-Triggered Meiotic Resumption in Oocytes by Enhancing an All-Or-None Positive Feedback Kinase Loop,” J. Biol. Chem. 281(51):39455-39464 (2006); Woodrow et al., “Ras-Induced Serine Phosphorylation of the Focal Adhesion Protein Paxillin is Mediated by the Raf->MEK->ERK Pathway,” Exp. Cell Res. 287(2):325-338 (2003); Cai et al., “Glycogen Synthase Kinase 3- and Extracellular Signal-Regulated Kinase-Dependent Phosphorylation of Paxillin Regulates Cytoskeletal Rearrangement,” Mol. Cell. Biol. 26(7):2857-2868 (2006); Ishibe et al., “Paxillin Serves as an ERK-Regulated Scaffold for Coordinating FAK and Rac Activation in Epithelial Morphogenesis,” Mol. Cell. 16(2):257-267 (2004); Ishibe et al., “Phosphorylation-Dependent Paxillin-ERK Association Mediates Hepatocyte Growth Factor-Stimulated Epithelial Morphogenesis,” Mol. Cell. 12(5):1275-1285 (2003), which are hereby incorporated by reference in their entirety) indicate that Erk itself may directly phosphorylate paxillin at these serine residues.

To summarize, paxillin regulates Erk-mediated processes by two means: (1) by specifically regulating receptor-tyrosine kinase-mediated Erk1/2 activation via Src-mediated tyrosine phosphorylation, and (2) more broadly by regulating of Erk-mediated downstream processes like intra-nuclear transcription and proliferation via Erk-mediated serine phosphorylation (see FIG. 9C). Thus, paxillin can be both an affector and an effector of Erk signaling in prostate cancer cells, depending upon the stimulus. How paxillin acts as an effector to regulate transcription in prostate cancer cells is unknown; however, it is believed is that paxillin constitutively binds to inactive Erk1/2 to keep it sequestered. Consistent with this belief, the binding affinity of inactive Erk1/2 to paxillin appears higher than that of activated Erk1/2 (Ishibe et al., “Phosphorylation-Dependent Paxillin-ERK Association Mediates Hepatocyte Growth Factor-Stimulated Epithelial Morphogenesis,” Mol. Cell. 12(5):1275-1285 (2003); Lidke et al., “ERK Nuclear Translocation is Dimerization-Independent but Controlled by the Rate of Phosphorylation,” J. Biol. Chem. 285(5):3092-3102 (2010), which are hereby incorporated by reference in their entirety). Irrespective of how it is triggered, activated Erk1/2 is believe to then promote serine phosphorylation of paxillin, releasing Erk from paxillin and permitting Erk-mediated transcription, cell proliferation, migration, and invasion. Whether the details of this pathway may be refined further, the preceding examples underscore the importance of paxillin in regulating proliferation in both androgen-dependent and -independent prostate cancer regardless of the stimulus (steroids or other growth factors). Given its critical importance in regulating Erk-mediated prolif-eration, both the (total) expression level of paxillin and the nuclear translocation of serine-phosphorylated paxillin represent two key markers of prostate cancer proliferation (and, hence, tumor aggressiveness). Furthermore, the results of siRNA-mediated paxillin knockdown demonstrates that abrogation of paxillin in prostate cancer cells is a viable therapeutic approach for the treatment of androgen-dependent and -independent prostate tumors.

### Example 9

### Paxillin is Overexpressed in Human Prostate Cancer In Vivo

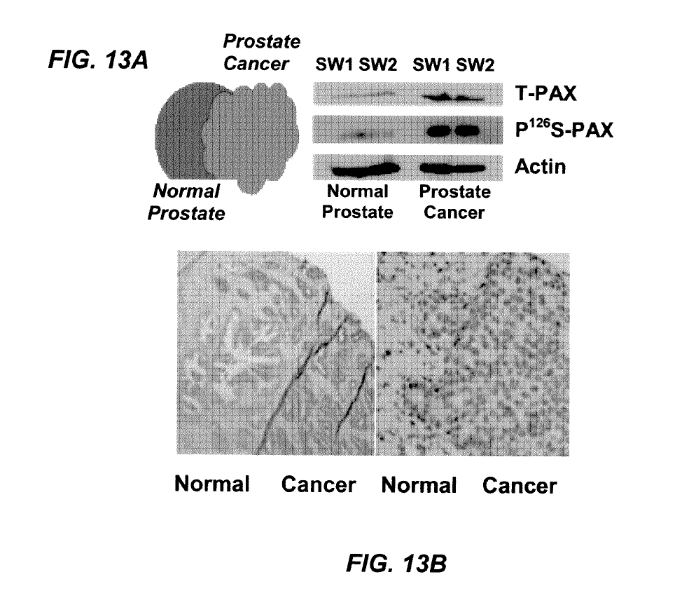

Since paxillin regulates prostate cancer cell proliferation in vitro, it was believed that paxillin also would be overexpressed in prostate cancer in vivo. Western blot and immunohistochemistry of patient samples reveals high expression of paxillin and phosphoserine-paxillin in cancer relative to adjacent normal prostate (FIGS. 13A-B). Combined with the aforementioned in vitro data, these results confirm the role that paxillin plays in prostate cancer growth and proliferation. Furthermore, the in vivo observation that, as in the prostate cancer cell lines, activated phosphoserine-paxillin is translocated to the nucleus supports the model illustrated in FIG. 9C, where Erk-mediated phosphorylation permits translocation of phosphoserine-paxillin to the nucleus where it enhances transcription and promotes tumor growth.

### Example 10

### Paxillin and Steroid-Triggered Transcription in Prostate Cancer Cells

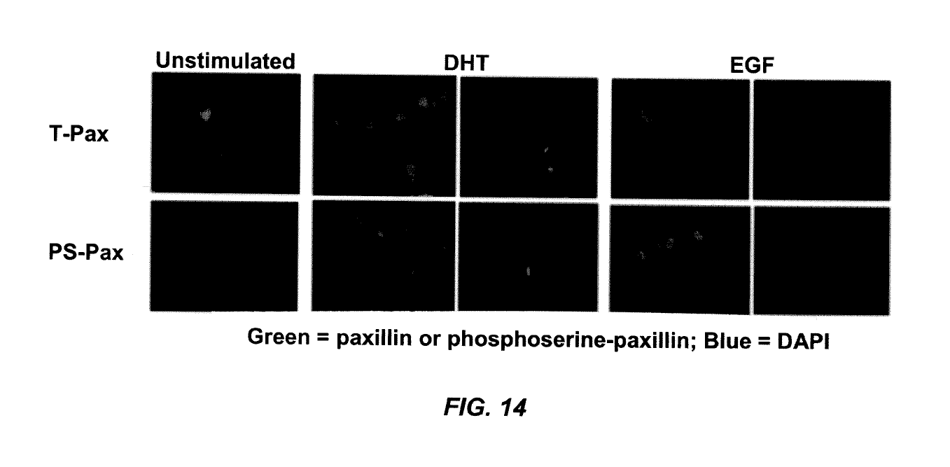

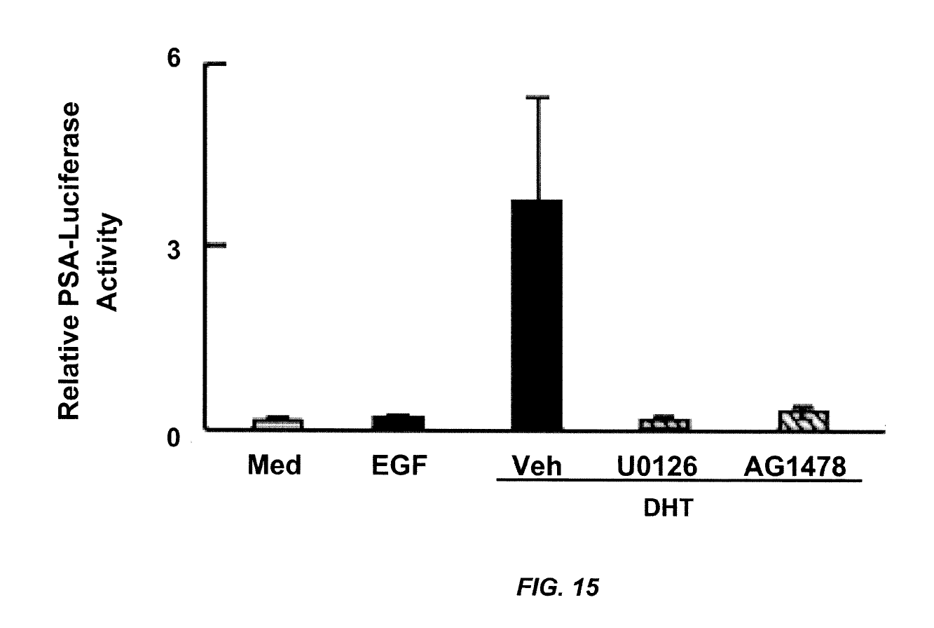

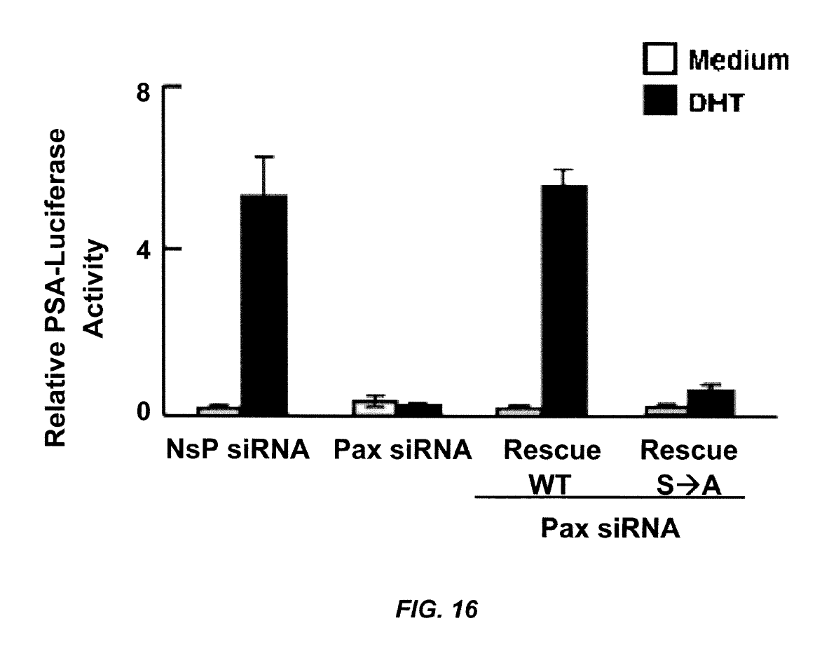

The subcellular localization of paxillin versus phosphoserine-paxillin was first examined. As expected, paxillin is localized primarily in the cytoplasm in resting cells (FIG. 14, unstimulated). Upon stimulation with DHT or EGF, some total paxillin (T-Pax) relocalizes the nucleus (FIG. 14, top panels), and nearly all phosphoserine-paxillin (PS-PAX) is in the nucleus (FIG. 14, bottom panels). This confirms the model of FIG. 9C, which posits that phosphoserine-paxillin regulates nuclear processes. Therefore, a common AR-mediated transcriptional event was examined: expression of PSA mRNA. It was found that DHT-induced expression of the PSA mRNA (by quantitative PCR) or activation of the PSA promoter (by luciferase assay) is dependent on both EGFR and MEK signaling, as inhibitors of either kinase blocked both processes (FIGS. 9A and 15). Furthermore, knockdown of endogenous paxillin in LnCAP cells prevented DHT-induced induction of PSA mRNA expression and promoter activity (FIGS. 9A and 15). Notably, expression of wild-type paxillin in these knockdown cells restored DHT-induced PSA promoter activity, while the paxillin mutant lacking the Erk-targeted serine residues (S→A) did not (FIG. 16). These results confirm that Erk-mediated phosphorylation of paxillin is required for AR mediated transcription in the nucleus. Together, these studies further emphasize that paxillin regulates cross-talk between extranuclear kinase signaling and intranuclear transcription.

### Example 11

### Paxillin Regulates AR Localization to the Nucleus

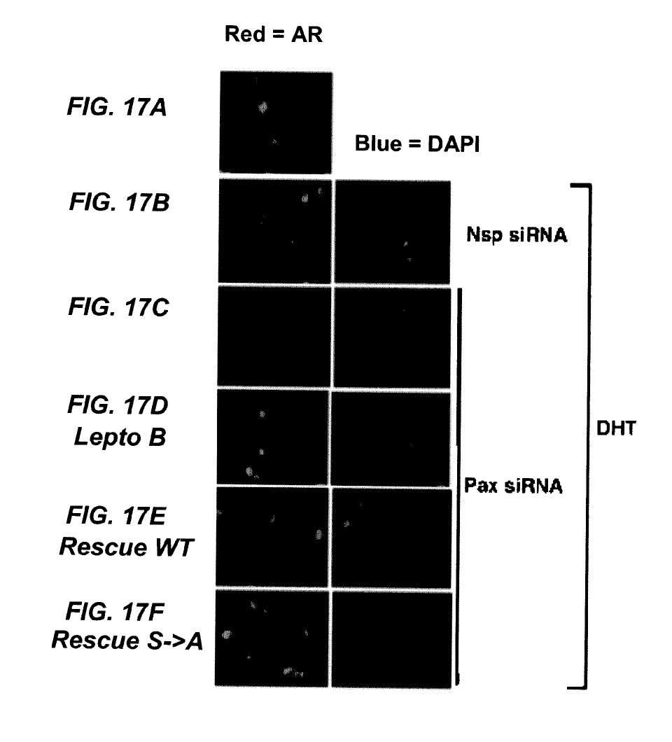

Cellular staining was carried out using fluorescence imaging for androgen receptors (AR) and DAPI. These data indicated that AR localization in the nucleus requires paxillin, as knockdown of endogenous paxillin abrogates DHT-induced nuclear localization of the AR (FIG. 17C versus 17A-B). Furthermore, knockdown and rescue experiments confirm that phosphoserine-paxillin is the moiety regulating AR translocation to the nucleus, since the mutant lacking the Erk serine targets (S A) cannot sustain AR nuclear localization (FIG. 17F versus 17E). Importantly, leptomycin B, which prevents nuclear export through CRM, rescues the nuclear localization of the AR in paxillin-knockdown cells stimulated with DHT (FIG. 17D). This result confirms that, upon DHT stimulation, the AR cycles between the cytoplasm and the nucleus, and that phosphoserine-paxillin prevents nuclear export. The latter allows the AR to remain in the nucleus, bind DNA, and promote transcription.

### Example 12

### Paxillin Modulates More than Just AR-Mediated Transcription

The importance of paxillin in normal physiological functions is evident from global paxillin knock-out studies, demonstrating that ablation of paxillin in mice is embryonic lethal (Migliaccio et al., “Steroid-Induced Androgen Receptor-Oestradiol Receptor Beta-Src Complex Triggers Prostate Cancer Cell Proliferation,” EMBO J. 19(20):5406-5417 (2000); Suzuki et al., “Androgen Receptor Involvement in the Progression of Prostate Cancer,” Endocr. Relat. Cancer 10(2):209-216 (2003)). It also has previously been demonstrated that in Xenopus oocytes, paxillin is essential for non-genomic androgen-induced Erk signaling and subsequent Erk-mediated oocyte maturation (Rasar et al., “Paxillin Regulates Steroid-Triggered Meiotic Resumption in Oocytes by Enhancing an All-Or-None Positive Feedback Kinase Loop,” J. Biol. Chem. 281(51):39455-39464 (2006)). Specifically, paxillin is required for synthesis and activation of MOS (the germ cell Raf homolog), which then promotes MEK and subsequently Erk signaling (Rasar et al., “Paxillin Regulates Steroid-Triggered Meiotic Resumption in Oocytes by Enhancing an All-Or-None Positive Feedback Kinase Loop,” J. Biol. Chem. 281(51):39455-39464 (2006)). Interestingly, Erk-mediated phosphorylation of paxillin is also required for androgen-induced oocyte maturation. Thus, in oocytes as in prostate cancer cells, paxillin is both an affector and effector of Erk signaling.

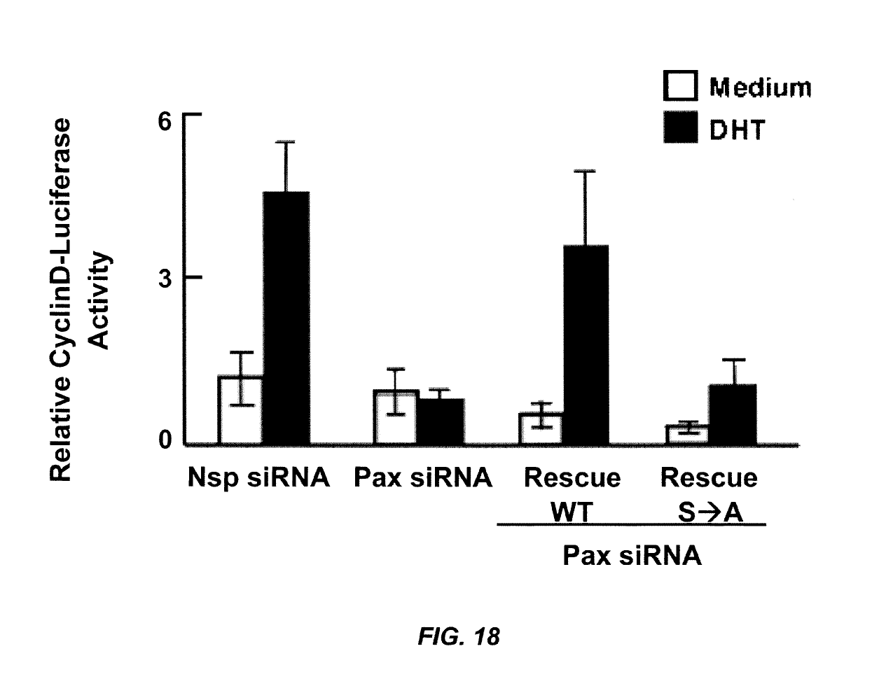

Cyclin D1 is activated in response to EGF and requires Erk signaling. To confirm that paxillin also mediates expression of Erk-dependent, AR-independent genes, cyclin D1 mRNA expression was measured in response to EGF (FIG. 9B, supra) as well as EGF-induced activation of the cyclin D1 promoter driving luciferase production (FIG. 18). Knockdown of endogenous paxillin and re-expression of the serine-mutated paxillin (S→A) does not rescue EGF-mediated cyclin D1 promoter activity (FIG. 18), confirming that Erk-mediated phosphorylation of paxillin is necessary for cyclin D1 promoter activity.

### Example 13

### Generation and Testing of Prostate Cancer Cell-Specific Chimeric Aptamers

A chimeric RNA molecule will be prepared using the PSMA-binding aptamer A10 (SEQ ID NO: 15) or A10-3 (SEQ ID NO: 16) in combination with one or more distinct paxillin shRNA (constructed using the siRNA of SEQ ID NOS: 1-14) using the procedures of Ni et al., “Prostate-targeted Radiosensitization via Aptamer-shRNA Chimeras in Human Tumor Xenografts,” J. Clin. Invest. 121(6):2383-2390 (2011). Androgen-independent PC3 and androgen-dependent LnCAP cells will be treated with 1 nM, 10 nM, 100 nM, and 500 nM of the chimera using HiPerFect transfection reagent (Qiagen) for 72 hours, serum-starved overnight, and stimulated with ethanol, DHT (25 nM), or EGF (20 ng/ml) for 24 hours. Proliferation of the cells will be assessed by MTT assay as described in the preceding examples.

In addition, cells will be plated 2000 cells per plate, and 24 hours later the cells will be irradiated with 6 Gy using a ¹³⁷Cs irradiator at approximately 0.6 Gy/min. Cell viability will be assessed after 12 days. This will indicate whether the combination of chimera therapy and radiotherapy causes greater inhibition of cancer cell survival.

Chimeras successful in vitro will also be screened using a xenograft animal model. 8-week old athymic nude mice will be inoculated with 5×10⁶ PC3 or LNCaP cells subcutaneously, and tumors will be grown to at least 0.8 cm in diameter. For aptamer-shRNA chimera knockdown of paxillin, tumors will be directly injected with 200 pmol or 400 pmol on days −7 and −6. On day 0, tumors will be harvested from one cohort of mice, and partitioned for RNA extraction of formalin fixation and IHC analysis. On day 0, a second cohort will receive 6 Gy of local IR (˜5.8 Gy/min) to the tumor site. Tumors will be measured every two days until four-times initial tumor volume is achieved in control animals.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of assessing aggressiveness or proliferative activity of a cancer that is capable of both steroid-dependent and steroid-independent growth and proliferation, the method comprising:
obtaining a cancer sample from a patient; and
determining whether cancer cells in the sample display an increase in the expression of paxillin or an increase in paxillin serine-phosphorylation in comparison to a control.

2. The method according to claim 1, wherein the cancer is prostate cancer, testicular cancer, breast cancer, endometrial cancer, uterine cancer, or ovarian cancer.

3. The method according to claim 1, wherein the control is (i) the expression of paxillin or the level of paxillin serine-phosphorylation in a normal cell of the same type and tissue that is cancerous; or (ii) the expression of paxillin or the level of paxillin serine-phosphorylation in a non-aggressive cancer cell of the same type and tissue.

4. (canceled)

5. The method according to claim 1, wherein the increase in the expression of paxillin or the increase in paxillin serine-phosphorylation is determined by immunoassay.

6-7. (canceled)

8. The method according to claim 1, wherein the increase in the level of paxillin serine-phosphorylation is determined.

9. The method according to claim 1, wherein both the expression level of paxillin and the level of paxillin serine-phosphorylation are determined.

10. The method according to claim 1 further comprising:
obtaining a second cancer sample from the patient; and
determining whether cancer cells in the second sample display an increase in the expression of paxillin or an increase in paxillin serine-phosphorylation in comparison to a control and/or in comparison to the first sample.

11. The method according to claim 10, wherein said obtaining a second cancer sample occurs following a delay of at least 7 days following said obtaining a cancer sample.

12. The method according to claim 10, wherein said obtaining a second cancer sample occurs following administration of a treatment protocol to the patient.

13. A method of treating cancer comprising:
administering to a patient having cancer an amount of an agent that inhibits paxillin expression or activity of serine-phosphorylated paxillin in the cancer cells, whereby said administering is effective to treat the cancer.

14. The method according to claim 13, wherein the cancer is capable of both steroid-dependent and steroid-independent growth and proliferation.

15. The method according to claim 13, wherein the cancer is prostate cancer, testicular cancer, breast cancer, endometrial cancer, uterine cancer, or ovarian cancer.

16. The method according to claim 13, wherein the agent inhibits paxillin expression.

17. The method according to claim 16, wherein the agent comprises RNAi.

18-19. (canceled)

20. The method according to claim 13, wherein the agent inhibits paxillin phosphorylation at Ser-83 or Ser-126 or Ser-130.

21. The method according to claim 20, wherein the agent is a nucleic acid aptamer or an anti-paxillin antibody or antibody fragment that binds to non-phosphorylated paxillin and prevents phosphorylation thereof.

22-23. (canceled)

24. The method according to claim 13, wherein the agent is targeted for cellular uptake by cancer cells expressing a cancer cell surface marker.

25-27. (canceled)

28. The method according to claim 24, wherein the cancer is prostate cancer and the cancer cell surface marker is prostate membrane specific antigen.

29. The method according to claim 24, wherein the cancer is breast, ovarian, endometrial, uterine, or testicular cancer and the cancer cell surface marker is Her-2 or Her-3.

30. A therapeutic agent comprising:
a first molecule that inhibits paxillin expression or activity of serine-phosphorylated paxillin, which first molecule is linked directly or indirectly to a second molecule that binds specifically to a cell surface marker of a cancer cell.

31. The therapeutic agent according to claim 30, wherein the cell surface marker is for a prostate, testicular, breast, ovarian, uterine, or endometrial cancer cell.

32. The therapeutic agent according to claim 30, wherein the cell surface marker is prostate membrane specific antigen, Her-2, or Her-3.

33. (canceled)

34. The therapeutic agent according to claim 30, wherein the first molecule is an RNAi that inhibits expression of paxillin.

35. The therapeutic agent according to claim 30, wherein the first molecule is an aptamer or an antibody or binding fragment thereof that interferes with phosphorylation of paxillin at one or more of serine residues Ser-83, Ser-126, and Ser-130.

36. The therapeutic agent according to claim 30, wherein the first and second molecules are either covalently linked together or linked by an affinity ligand pair.

37-39. (canceled)

40. A pharmaceutical composition comprising a carrier and a therapeutic agent according to claim 30.
